(19) United States
(12) Patent Application Publication
(10) Pub. No.: US 2016/0199231 A1
(43) Pub. Date: Jul. 14, 2016

(54) CLOSURE DEVICES AND METHODS FOR BIVALVED CASTS

(71) Applicant: Boa Technology, Inc., Denver, CO (US)

(72) Inventor: James Capra, Steamboat Springs, CO (US)

(73) Assignee: Boa Technology, Inc., Denver, CO (US)

(21) Appl. No.: 14/993,865

(22) Filed: Jan. 12, 2016

Related U.S. Application Data
(60) Provisional application No. 62/102,468, filed on Jan. 12, 2015.
(60) Provisional application No. 62/150,713, filed on Apr. 21, 2015.

(51) Int. Cl. A61F 13/04; A61F 5/01; A61F 5/058

(52) U.S. Cl. CPC A61F 13/048; A61F 5/05866; A61F 5/0585; A61F 5/0106; A61F 5/0118; A61F 5/0111

(57) ABSTRACT

A device for adjusting a fit of a brace about a limb includes a tension member and a tightening mechanism. The tightening mechanism is operably coupled with the tension member so that the tension member forms a loop. The loop is positionable about the brace to circumferentially surround an outer surface of the brace. The tightening mechanism is operable to decrease a diameter of the loop so that, after the loop is positioned about over the brace, the tension member constricts about the outer surface of the brace to tighten the brace about the limb.

### CROSS-REFERENCE TO RELATED APPLICATIONS

This application claims priority to Provisional U.S. patent application Ser. No. 62/102,468 filed Jan. 12, 2015, entitled “Closure Devices and Methods for Bivalved Casts” and Provisional U.S. patent application Ser. No. 62/150,713 filed Apr. 21, 2015, entitled “Closure Devices and Methods for Bivalved Casts.” The entire disclosure of both of the aforementioned Provisional U.S. Patent Applications are hereby incorporated by reference, for all purposes, as if fully set forth herein.

### BACKGROUND OF THE INVENTION

This invention relates generally to closure devices and more specifically to closure devices that may be employed with braces to adjust the fit of the brace about a limb.

Braces are fit about limbs to provide various therapeutic treatments. For example, casts are commonly positioned and secured over broken limbs to aid in healing of the broken bones. Such braces, and in particular casts, need to be securely fit about the limb to properly treat the limb, but should not be overly tight so that the patient is not comfortable. A problem with some braces, and in particular casts, is that the fit of the braces are not adjustable. As such, overly tight braces are uncomfortable to wear while overly loose braces may not provide the necessary degree of support. Bivalved casts help address some of these issues by allowing the casts to open or flex to some degree, but various issues still remain with these types of casts. The embodiments described herein enable the fit of braces, and in particular casts, to be quickly and conveniently adjusted to provide needed support and comfort.

### BRIEF DESCRIPTION OF THE INVENTION

The embodiments herein enable a physician and/or user to adjust the fit of a brace about a limb. According to one aspect, a device for adjusting a fit of a brace about a limb includes a tightening mechanism (e.g., the reel assembly described herein below) that includes a housing having an interior region, a spool that is disposed within the interior region of the housing, and a knob that is operably coupled with the spool. The spool is rotatable within the interior region of the housing and the knob is coupled with the spool so that rotation of the knob in a first direction causes the spool to rotate within the interior region of the housing in the first direction. The device also includes a tension member that is coupled with the spool so that rotation of the spool in the first direction causes the tension member to be wound about the spool. The tension member forms a loop that is positionable about the brace to circumferentially surround an outer surface of the brace. Winding of the tension member about the spool causes a diameter of the loop to decrease such that, after the loop is positioned about over the brace, the tension member constricts about the outer surface of the brace to tighten the brace about the limb. In some embodiments, the tension member is not coupled with any guide that is attached to the brace that guides or directs the tension member about a path.

The device may also include a guide member that is coupled directly with the tightening mechanism to redirect the tension member and thereby define a second loop that is also positionable about the brace to circumferentially surround the outer surface of the brace. The tension member may be slidably disposed within a channel of the guide member so that winding of the tension member about the spool causes a diameter of the second loop to decrease simultaneously with the decrease in the diameter of the loop. A first end of the tension member may be coupled with the spool and a second end of the tension member may be adjustably coupled with the housing in a manner that allows the diameter of the loop to be decreased without winding the tension member about the spool.

A bottom surface of the housing that is opposite the knob may include an adhesive that is bondable to an outer surface of the brace. In some instance, the tightening mechanism may be attachable to a base component that is positioned under an outer layer of the brace, such as under one or more layers or wrappings of gauze. The tightening mechanism may be configured so that rotation of the knob in the first direction causes the spool to incrementally rotate in the first direction and thereby incrementally tighten the brace about the limb, and so that a rotation of the knob in a second direction causes the spool to incrementally rotate in the second direction and thereby incrementally loosen the brace about the limb. In some instances, the tightening mechanism may not operable to allow the spool to freely rotate within the interior region of the housing. In other instances, the tightening mechanism may be operable so that the spool is freely rotatable within the interior region of the housing.

According to another aspect, a device for adjusting a fit of a brace about a limb includes a tension member and a tightening mechanism that is operably coupled with the tension member so that the tension member forms a loop that is positionable about the brace to circumferentially surround an outer surface of the brace. The tightening mechanism is operable to decrease a diameter of the loop so that, after the loop is positioned about over the brace, the tension member constricts about the outer surface of the brace to tighten the brace about the limb.

In some instances, the tension member is uncoupled from any guide that is attached to the brace that guides or directs the tension member about a path. The device may further include a guide member that is coupled with the tightening mechanism to redirect the tension member and thereby define a second loop that is also positionable about the brace to circumferentially surround the outer surface of the brace. The tension member may be slidably disposed within a channel of the guide member so that operation of the tightening mechanism causes a diameter of the second loop to decrease simultaneously with the decrease in the diameter of the loop. One end of the tension member may be adjustably coupled with the tightening mechanism in a manner that allows the diameter of the loop to be decreased without operation of the tightening mechanism. A bottom surface of the tightening mechanism may include an adhesive that is bondable to an outer surface of the brace.

The tightening mechanism may be configured so that a first operation of the tightening mechanism decreases the diameter of the loop and thereby incrementally tightens the brace about the limb and so that a second operation of the tightening mechanism increases the diameter of the loop and thereby incrementally loosens the brace about the limb. The tightening mechanism may include a knob that is rotatable in a first direction to decrease the diameter of the loop and that is rotatable in a second direction to increase the diameter of the loop.

According to another aspect, a method of adjusting a fit of a brace about a limb is provided. The method is applicable to a device that includes a tension member and a tightening mechanism that is operably coupled with the tension member with the tension member forming a loop. The method includes positioning the loop about the brace so that the tension member circumferentially surround an outer surface of the brace and operating the tightening mechanism to decrease a diameter of the loop and thereby constrict the tension member about the outer surface of the brace to tighten the brace about the limb.

In some instances, one end of the tension member may be adjustably coupled with the tightening mechanism. In such instances, the method may also include adjusting the one end of the tension member relative to the tightening mechanism so that the diameter of the loop is decreased without operation of the tightening mechanism. In some instances, the method may further include performing a first operation of the tightening mechanism to decrease the diameter of the loop and thereby incrementally tighten the brace about the limb and/or performing a second operation of the tightening mechanism to increase the diameter of the loop and thereby incrementally loosen the brace about the limb. In such instances, performing the first operation of the tightening mechanism may include rotating a knob in a first direction and performing the second operation of the tightening mechanism may include rotating the knob in a second direction.

The device may also include a guide member that is coupled with the tightening mechanism to redirect the tension member and thereby define a second loop that is also positionable about the brace to circumferentially surround the outer surface of the brace. The tension member may be slidably disposed within a channel of the guide member so that operation of the tightening mechanism causes a diameter of the second loop to decrease simultaneously with the decrease in the diameter of the loop.

### BRIEF DESCRIPTION OF THE DRAWINGS

The present invention is described in conjunction with the appended figures:

FIGS. 1 & 2 illustrate an embodiment of a tensioning system that may be integrated with a cast or brace.

FIGS. 3-5 illustrate exemplary components of a tensioning system for use with a cast or brace.

FIGS. 6 & 7 illustrate embodiments of multiple components on a single panel for use in with a cast or brace.

FIGS. 8 & 9 illustrate embodiments of a coupling mechanisms that may be used to couple a reel with a panel.

FIGS. 10 & 11 illustrate a lace tensioning system being fit about and/or coupled with a bivalved cast.

FIG. 12 illustrates a panel coupled with a bivalve cast in a manner that allows a wider area of the cast to be easily tensioned via operation of a single reel.

FIG. 13 illustrates alternative guides that are formed into or otherwise coupled with a cast and that are used to guide or direct a lace about a path along the cast.

FIGS. 14A-C illustrate embodiments of the guides of FIG. 13.

FIGS. 15-17 illustrate embodiments of tensioning systems employed on a leg cast or brace.

FIG. 18 illustrates a tensioning system having a portion of a lace positioned over a window of a cast or brace in order to maintain the window in a closed position.

FIGS. 19-20B illustrate various embodiments of tensioning systems employed on bivalved casts or braces.

FIG. 21 illustrates a compliance cap that may be secured to a reel to limit or restrict a user's access to and/or operation of the reel.

FIG. 22 illustrates a shield that locks or otherwise prevents a reel from being accessed and/or adjusted by a user.

FIGS. 23-25 illustrate another exemplary embodiment of a tightening mechanism for use with a brace or cast.

FIG. 26 illustrates the tightening mechanism of FIGS. 23-25 positioned about a bivalve cast.

FIG. 27 illustrates an exemplary packaging for the tightening mechanism of FIGS. 23-25.

FIGS. 28A-B illustrate a method of using the tightening mechanism of FIGS. 23-25 with a bivalve cast.

FIGS. 29A-C illustrate other embodiments of a housing of a tightening mechanism.

FIGS. 30A-B illustrate an embodiment of coupling a tightening mechanism with a body of a bivalve cast.

FIGS. 31A-34 illustrate another embodiment of coupling a tightening mechanism with a bivalve cast.

FIG. 35 illustrates an embodiment of a tightening component that may be integrally formed with a bivalve cast.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION OF THE DRAWINGS

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth in the appended claims.

The embodiments described herein provide tensioning components that may be easily integrated into materials of a bivalved cast or formed into and/or secured on a bivalved brace. A bivalved casts is a cast that is often cut in half, or cut multiple times, to relieve pressure on a limb positioned within the cast. The cast may be formed of a single cast component having a single longitudinal cut, or may be formed via two or more longitudinal cuts that divide the cast into two components. Such casts may be especially important when an individual has decreased sensation, or essentially no sensation, in the portion of the body that is surrounded by the cast. The casts are often formed from layers of wrapped moistened gauze, such as gauze treated with fiberglass solutions and/or resins. The layers are allowed to dry and harden and then the cast is cut. Traditional bivalve casts may be tightened about a limb by wrapping additional layers of often untreated gauze over the bivalve cast. Pressure may be relieved from the limb by unwrapping the gauze from the bivalve cast. Constant wrapping and unwrapping of the gauze from the bivalve cast is often burdensome and time consuming. Further, the use of gauze does not allow the tightness of the bivalve cast to be easily adjusted.

The embodiment presented herein provide efficient and easy means of tightening and untightening a bivalve cast positioned about a limb. The embodiments also provide an easy means of adjusting the pressure of the bivalve cast about a limb. The ability to tighten and untighten a bivalve cast about a limb, and to adjust the tightness of the bivalve cast, is achieved via a tightening mechanism that tensions a tension member, such as a lace, cord, strap, and the like. The tension member may be positioned about the bivalve cast so that tensioning and loosening of the tension member effects tightening or untightening of the bivalve cast about the limb.

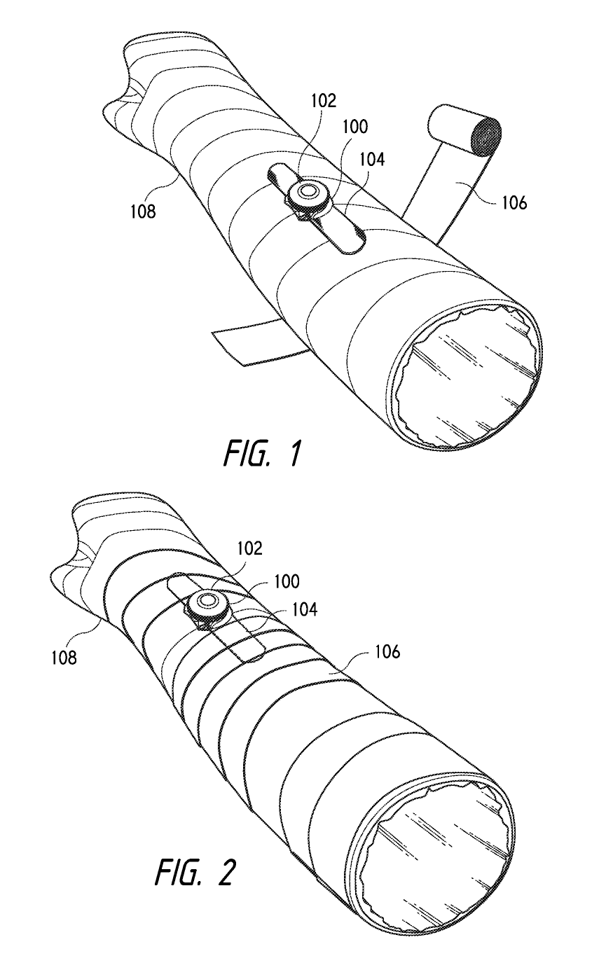

Referring now to the various figures, FIGS. 1 and 2 illustrate one embodiment of a tensioning system that may be integrated with a bivalve cast or brace. FIG. 1 shows a tensioning system 100 including a tensioning device or reel 102, such as described in U.S. patent application Ser. No. 14/071,435, filed Nov. 4, 2013, and entitled “Coupling Members for Closure Devices and Systems,” the entire contents of which are hereby incorporated by reference. Other exemplary embodiments of reel assemblies are further described in U.S. Pat. No. 8,468,657, filed Nov. 20, 2009, entitled “Reel Based Lacing System,” in U.S. patent application Ser. No. 14/328,521, filed Jul. 10, 2014, entitled “Closure Devices Including Incremental Release Mechanisms and Methods Therefore,” and in U.S. patent application Ser. No. 14/487,024, filed Sep. 15, 2014, entitled “Failure Compensating Lace Tension Devices and Methods,” the entire disclosures of which are incorporated by reference herein.

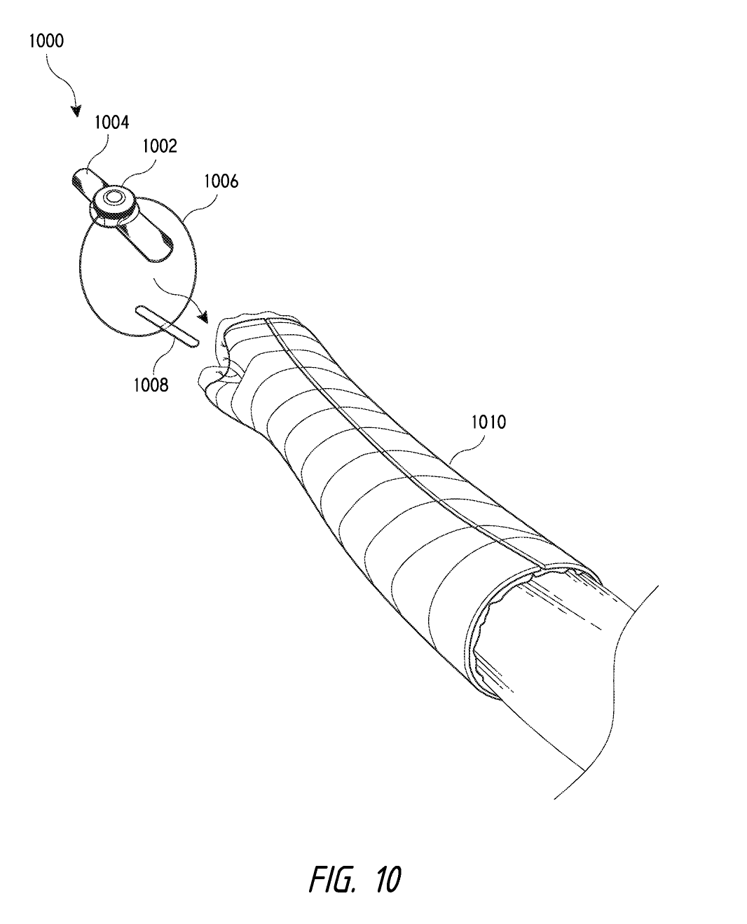

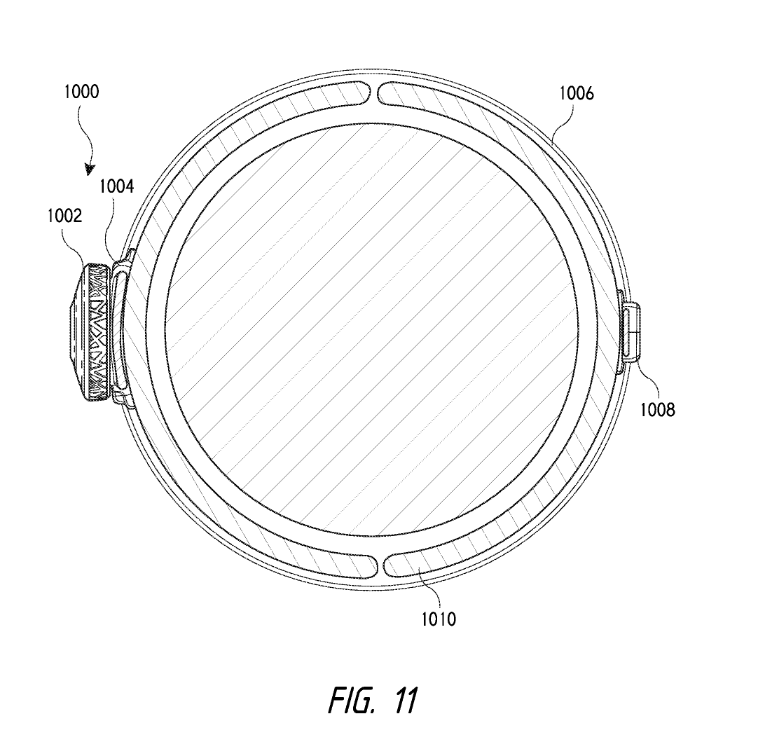

The tensioning system includes flanged portions 104 that extend longitudinally along the bivalve casts. In some embodiments, the flanged portions 104 may extend parallel to the cut or cuts formed on or in the bivalved cast 108 (see FIG. 10 for an exemplary cut). Flanged portions 104 may be formed from any material, such as materials that may adhere to a fiberglass solution of the gauze. This orientation minimizes deformation of the flanged portions 104 when secured to a limb, and also enables a tensioning system 100 to be adaptable to a number of shapes and sizes of body parts. The flanged portions 104 may be covered by layers of gauze 106 and/or other cast-forming materials to secure the tensioning system 100 onto cast 108 and/or make the tensioning system integral with the cast 108. After the gauze 106 has been wrapped around the tensioning system 100 and allowed to dry, the tensioning system 100 may allow a patient, physician or other medical personnel, and/or other user to apply tension to the cast 108 or to reduce tension in the cast. As shown in FIG. 2, after application of an outer layer of gauze 106, the tensioning system 100 may be integrally formed into the cast 108.

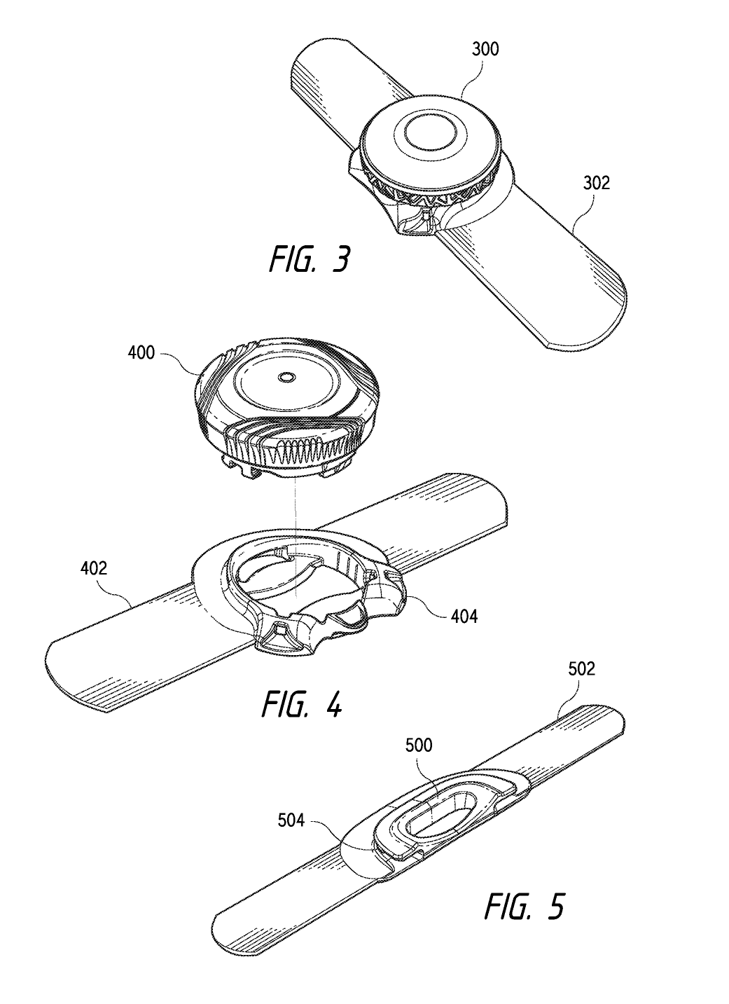

FIGS. 3-5 illustrate exemplary components of a tensioning system for use in a bivalved cast or brace application. FIG. 3 shows a reel 300 having a flanged portion 302 for securing the reel 300 to a bivalved cast as shown in FIGS. 1 and 2. Reel 300 may be used to tension a lace (not shown) to tighten and/or maintain a pressure of a cast or brace. Flanged portion 302 may be rigid or flexible. The flanged portion 302 may be integral with reel 300 or may be a separate component that is coupleable with the reel 300. FIG. 4 shows an embodiment of a reel 400 that is secured on flanged portion 402 via a coupling port or “bayonet” 404 that is formed with or secured to flanged portion 402. Bayonet 404 is configured for releasable attachment with reel 400. By using one or more bayonets 404 with releasable reels 400, reels 400 may be recycled for use with new patients and/or may be used when recasting an injury a second or subsequent time. Releasable reels, such as reel 400 coupled with bayonet 404, also prevent moving parts of the reel 400 from getting clogged with any of the wet solution used on the gauze during coupling of the flanged portion 402 with the cast or brace since the reel 400 may be attached to the bayonet 404 after the gauze dries.

FIG. 5 illustrates a guide 500 having a flanged portion 502. Flanged portion 502 may be formed integral with the guide 500 or may be secured via another process, such as using an adhesive or by radio frequency (RF) welding. Guide 500 may include an open back 504 such that a lace may be easily inserted into a channel of the guide 500 through open back 504 on a finished cast to complete a tensioning system. Thus, a reel, when turned, may cause a lace passing through guide 500 to be tightened, thereby pulling guide 500, and the portion of the cast/brace coupled with flanged portion 502, to be pulled in the direction of the laces exiting guide 500. This makes the tensioning system quick and efficient for tightening a cast after the cast is formed around a patient's limb. One or more guides may be positioned around a cast such that a lace may be used to create a tensioning system to apply pressure to the cast. By using separate, single components, spacing and alignment may be customized to fit a particular size and shape of the cast. In this manner, components need not be specially made and may be used to treat a variety of injuries using a variety of different casts/braces.

The reels described herein may be configured to adjust tension applied to a lace in different manners and/or increments. In some embodiments the reels may be adjusted by a user to release tension and accommodate swelling under the cast. For example, some reels may be configured to adjust lace lengths by 1 millimeter in either direction, thus adjusting an amount of tension applied. In these or other embodiments, a clutch and/or tension gauge may be included in the reel to prevent the reel from being over-tensioned. An initial tension can be set and/or the clutch may release tension if the tension level increases beyond a threshold due to swelling within a selected range. Reels with clutch devices are described in U.S. patent application Ser. No. 14/322,690, filed Jul. 2, 2014, entitled “Tension Limiting Mechanisms for Closure Devices and Methods Therefor,” the entire contents of which are hereby incorporated by reference.

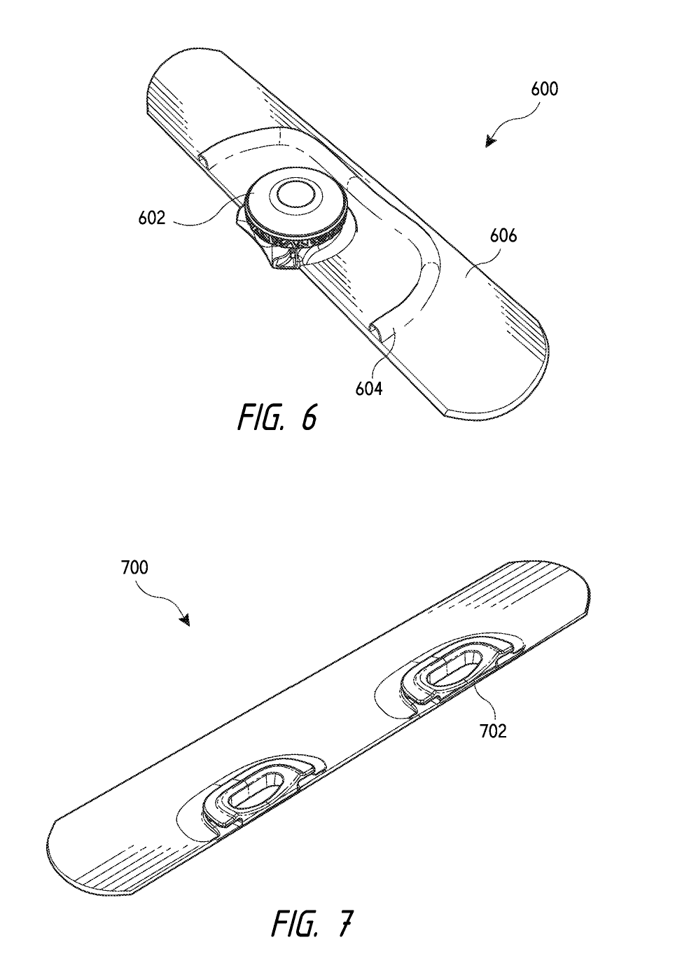

FIGS. 6 and 7 show embodiments of multiple components on a single panel for use in a bivalve cast or brace application. By incorporating multiple components onto a single panel, installation of various embodiments and configurations may be expedited during the formation of the cast. Additionally, the components on a single panel may be pre-aligned with each other, making installation simpler. In some embodiments, combinations of multiple component panels and single component panels may be used to form a particular desired tensioning system. FIG. 6 shows a panel 600 having a reel 602, a guide 604, and a flanged portion 606. In some embodiments, reel 602 may be removably coupled to the flanged portion 606, such as by using a bayonet as illustrated in FIG. 4. In other embodiments, reel 602 may be integrally formed with flanged portion 606. FIG. 7 shows a panel 700 having a pair of guides 702. In some embodiments, more than two guides 702 may be included on panel 700. Guides 702 may have a closed back, or an open back to allow easier lacing.

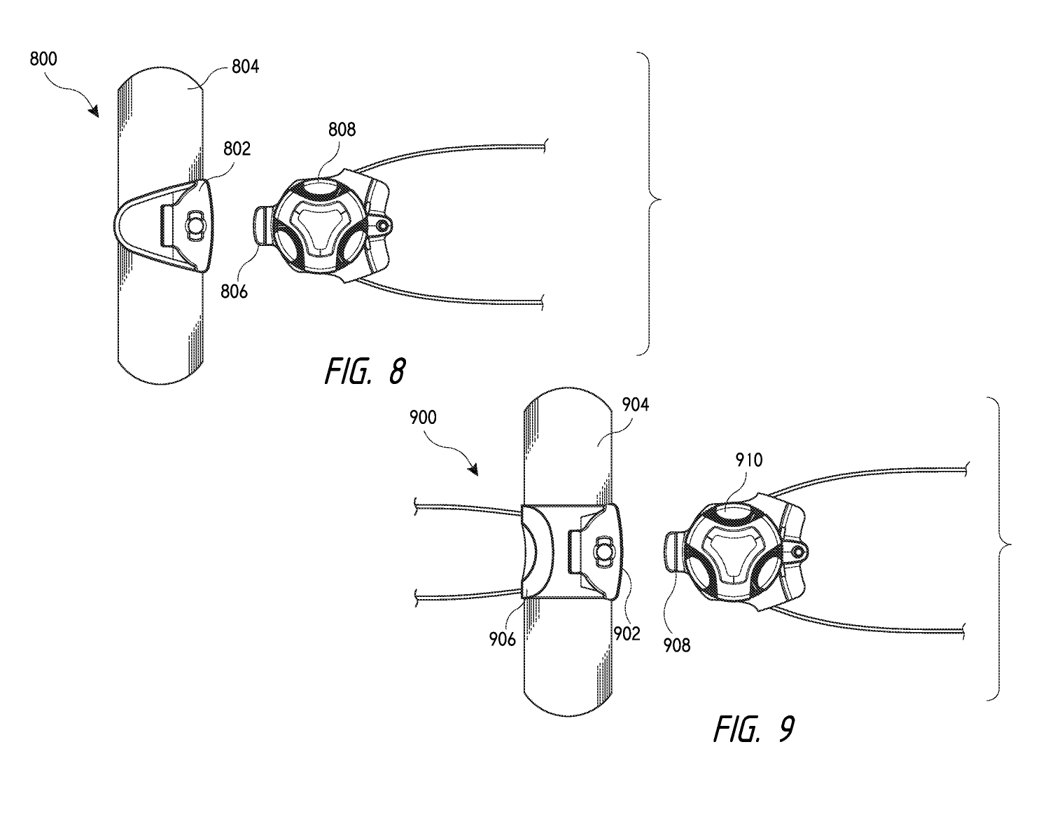

FIGS. 8 and 9 show alternative embodiments of coupling mechanisms to couple a reel to a panel. FIG. 8 shows a panel 800 having a snap receiver 802 and a flanged portion 804. A portion of a base 806 of reel 808 may be interfaced and secured within snap receiver 802, such as by snapping the portion of the base 806 into the snap receiver 802. The use of such a reel 808 and snap receiver 802 allows the reel 808 to be recycled for use in additional casts. FIG. 9 shows a panel 900 having a snap receiver 902 and a flanged portion 904. A guide 906 may be included on panel 900. For example, guide 906 may be formed integral with the snap receiver 902 and/or the flanged portion 904. In other embodiments, the guide 906 may be attached to the flanged portion 904 and/or the snap receiver 902. A portion of a base 908 of a reel 910 may be interfaced with the snap receiver 902 to secure the reel 910 to the panel 900 when the panel 900 is formed into a cast.

FIGS. 10 and 11 show a single lace tensioning system for a bivalved cast. FIG. 10 shows a tensioning system 1000 having a reel 1002, a flanged portion 1004, and a lace 1006. Lace 1006 may be wrapped around a cast 1010 formed around a user's limb. For example, the lace 1006 may wrap a cast 1010 positioned around a user's arm such as illustrated in FIG. 10. Lace 1006 may be made of any material, such as a textile, polyester, nylon coated steel, stainless steam, monofilament, or any other material that may provide and maintain sufficient tension within a tensioning system. A flanged guide 1008 may be included in tensioning system 1000. Flanged guide 1008 may be positioned opposite of the reel 1002 and flanged portion 1004 and may help prevent excessive pressure caused by the tensioning of the lace 1006 at a single position. In some embodiments, additional flanged guides may be used to further eliminate high tension points. In other embodiments, no flanged guides are used and the lace is in direct contact with the cast. FIG. 11 shows the tensioning system 1000 with lace 1006 tensioned around the cast 1010 and limb of the user. Both the flanged guide 1008 and flanged portion 1004 may be formed into the cast 1010 as gauze is wrapped around the limb.

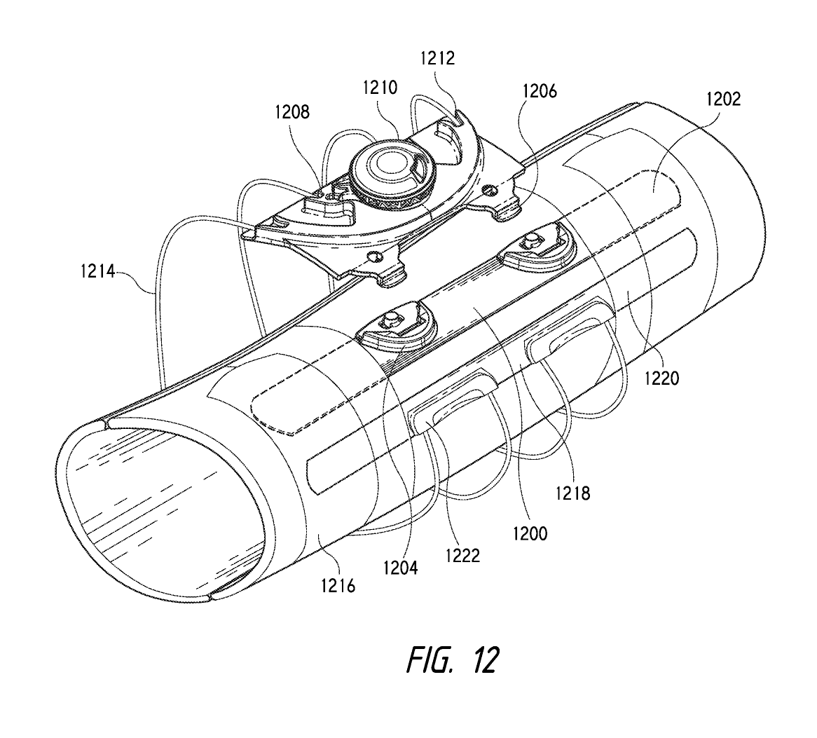

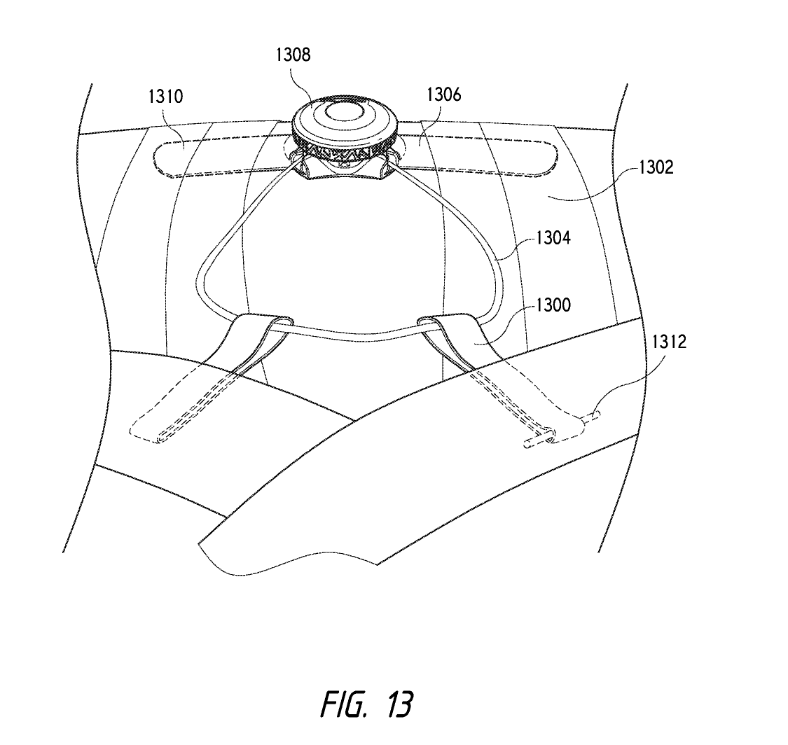

FIG. 12 depicts a panel 1200 having a flanged portion 1202 and multiple snap receivers 1204 to receive a portion of a base 1206 of a reel panel 1208. Panel 1200 may be formed into a cast 1216 to provide a base for the reel panel 1208. The reel panel 1208 may include a reel 1210 and one or more guides 1212 to direct a lace 1214. A panel 1218 may be included on an opposite side of the cast 1216 to complete a tensioning system for maintaining pressure in the cast 1216. Panel 1218 may include a flanged portion 1220 and one or more guides 1222 to direct lace 1214.

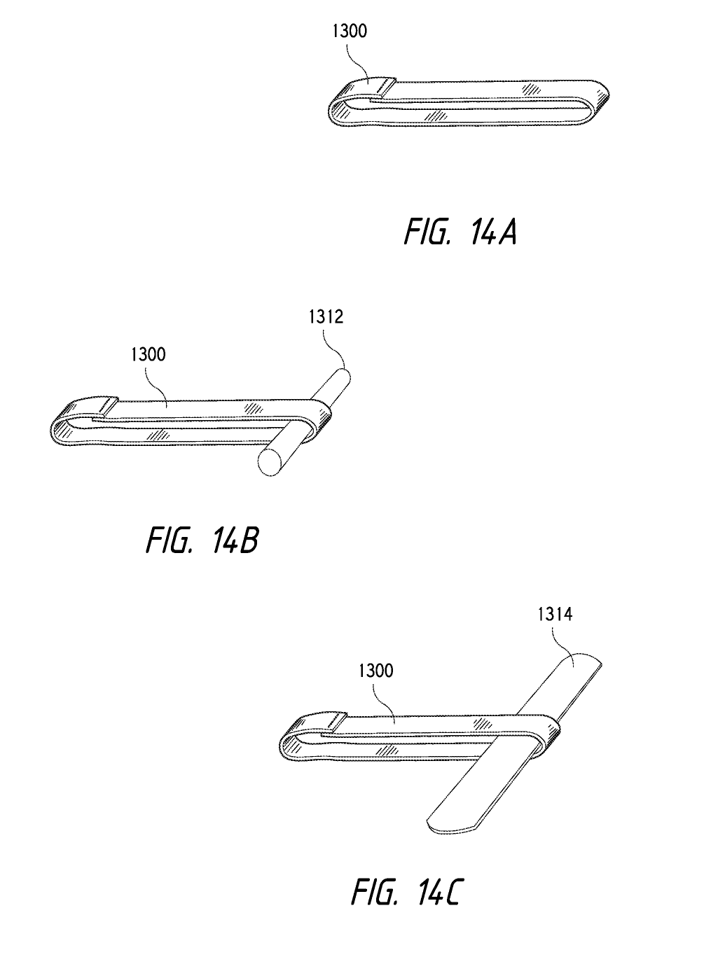

FIG. 13 illustrates “ghillie” guides 1300 formed into a cast 1302. Ghillie guides 1300 are formed from folding a strip of material backward and stitching the material together to form a channel through which a lace 1304 may be inserted. The material that is folded over and stitched together is typically fabric, but may also include plastic or other materials that may adhere to materials of the cast 1302. A portion of the ghillie guides 1300 may be covered in gauze during the formation of cast 1302 to secure the ghillie guides 1300 to the cast 1302. The dashed lines of the ghillie guides 1300 illustrate a portion of the guide 1300 that is covered and secured by gauze. In some embodiments, a panel or rod 1312 may be positioned within an opening of a covered portion of the ghillie guides 1300 to provide an additional load-bearing support. A panel 1306 may include a reel 1308 and a flanged portion 1310 that is formed into the cast 1302. The reel 1308 may tension a lace that is secured within the ghillie guides 1300 to form a tensioning system to apply pressure to the cast 1302. FIG. 14A shows a view of one ghillie guide 1300 prior to insertion into a cast. FIG. 14B shows a view of a rod 1312 inserted into an opening of one ghillie guide 1300 prior to insertion into a cast. FIG. 14C shows a panel 1314 inserted into an opening of one ghillie guide 1300 prior to insertion into a cast.

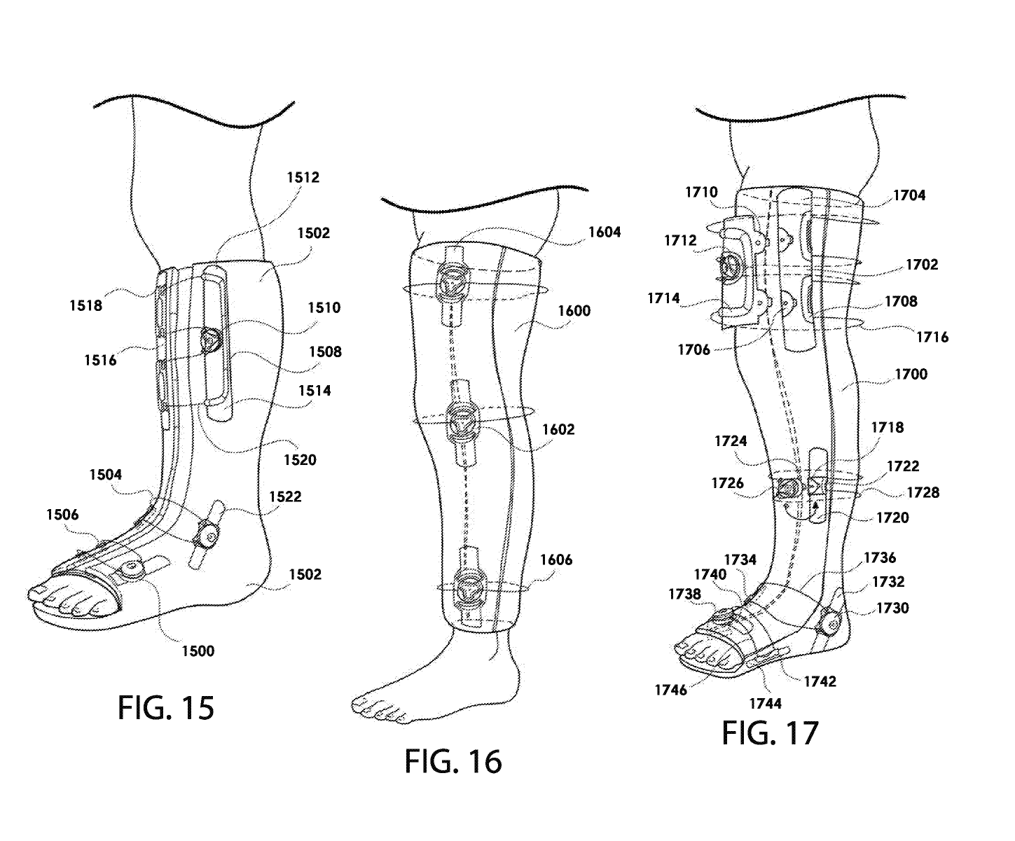

FIGS. 15-17 show embodiments of tensioning systems utilized on a leg cast. FIG. 15 shows a pair of reels 1500 with flanged portions 1522 secured to a lower portion of a cast 1502. Each reel 1500 tensions a lace 1504 that is interfaced with a guide 1506 having a flanged portion (not shown) positioned across a slit in the cast 1502 to form a tensioning system. A panel 1508 is also included on an upper portion of cast 1502. Panel 1508 includes a reel 1510, guide 1512, and flanged portion 1514 that may be formed into the cast 1502. A panel 1516 including multiple guides 1518 is positioned on an opposite side of the slit. A lace 1520 is tensioned by reel 1510 and is directed by guides 1512 and 1518 to form a tensioning system. Other numbers of guides and reels may be used to control pressure to the upper and lower portions of the cast 1502.

FIG. 16 shows a leg cast 1600 having a series of single lace reels 1602. Each single lace reel 1602 may be formed integral with a flanged portion 1604 or may be removably coupled with the flanged portion 1604, such as by using a bayonet as described herein. Flanged portion allows the single lace reels 1602 to be formed into the cast 1600. Each single lace reel 1602 may tension a single lace 1606 that wraps around a circumference of the cast 1600 to apply pressure. Although shown with three single lace reels 1602, any number of single lace reels may be used to apply a desired amount of pressure at a desired number of locations.

FIG. 17 shows a leg cast 1700 having a panel 1702 positioned along an upper portion of cast 1700. Panel 1702 includes a flanged portion 1704, snap receivers 1706, and guides 1708. Once formed into cast 1700, the snap receivers 1706 may receive a base 1710 of reel 1712 in a similar manner as shown in FIG. 8. Base 1710 may further include one or more guides 1714. Reel 1712 may be used to tension a lace 1716 that is directed by guides 1708 and 1714 to apply pressure around the upper portion of the cast 1700. A snap receiver 1718 having a flanged portion 1720 and a guide 1722, such as described in FIG. 9, may be formed into a medial portion of the cast 1700. A portion of a base 1724 of a reel 1726 may be inserted into snap receiver 1718 to secure the reel 1726 onto the cast 1700. Reel 1726 may tension a lace 1728 that is directed by guide 1722 to apply pressure to the medial portion of the cast 1700. A reel 1730 and flanged portion 1732 may be positioned near a bottom portion of the cast 1700. A guide 1734 and flanged portion (not shown) may be included on an opposite side of a slit in the cast 1700 such that a lace 1736 may be tensioned by reel 1730 and directed by guide 1734 to form a tensioning system. A reel 1738 and flanged portion 1740 may be positioned a top a foot portion of the cast 1700. Guides 1742 having flanged portions 1744 may be positioned on either side of the foot portion of the cast 1700. Reel 1738 may tension a lace 1746 that is directed by guides 1742 to form a tensioning system across the foot portion. It will be appreciated that other combinations and configurations of the guides and reels may be used to form tensioning systems to provide pressure in desired locations of casts for various body parts.

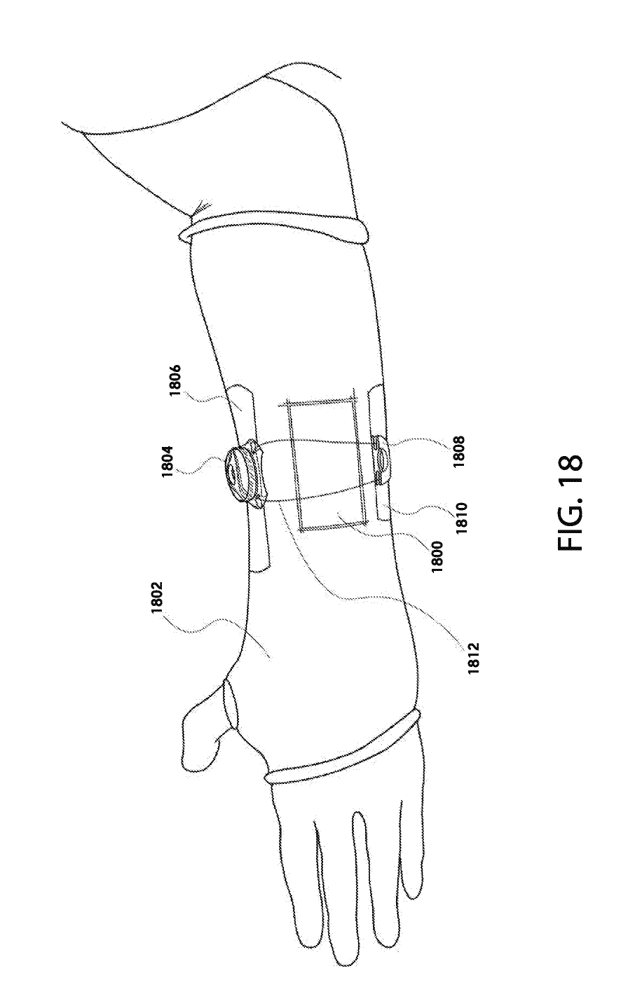

In some embodiments, a window may be cut into a cast to relieve pressure in a location due to swelling and/or to check on the progress of a healing injury. For example, in treatment of burn victims having broken limbs, a window may allow for the treatment of the burns while the cast still provides support for the broken limb to mend. These windows may need to be resealed with cast material to provide proper tension to the treatment site after the progress has been checked and/or any swelling reduces. FIG. 18 depicts a tensioning system used to close up a window 1800 in a cast 1802. A reel 1804 and flanged portion 1806 may be positioned on one side of window 1800 and a guide 1808 and flanged portion 1810 may be formed on an opposite side of window 1800. Reel 1804 may tension a lace 1812 that traverses the window 1800 to guide 1808 to apply pressure over material positioned over the window 1800.

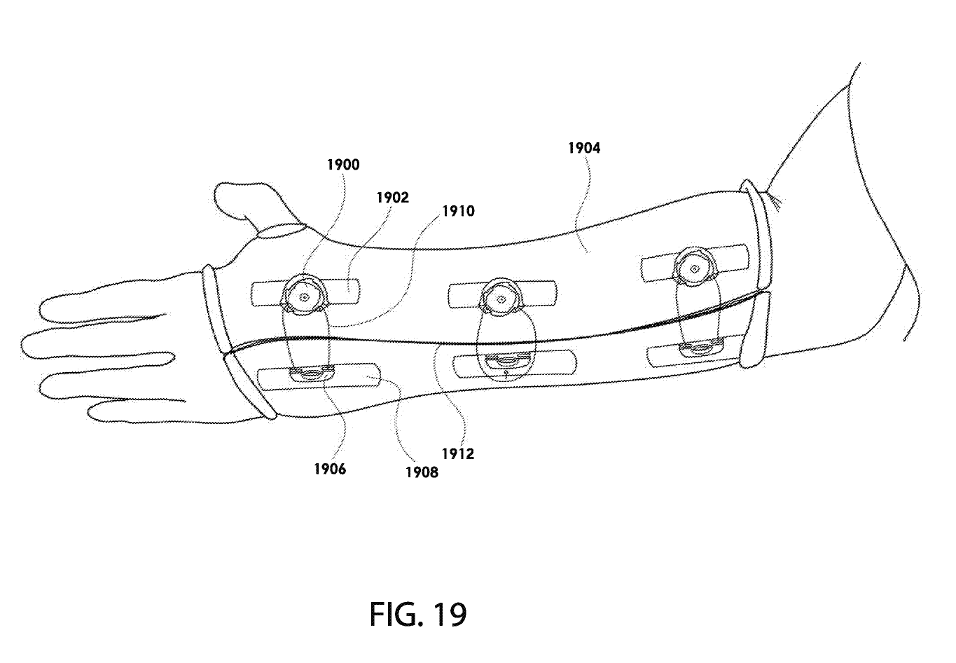

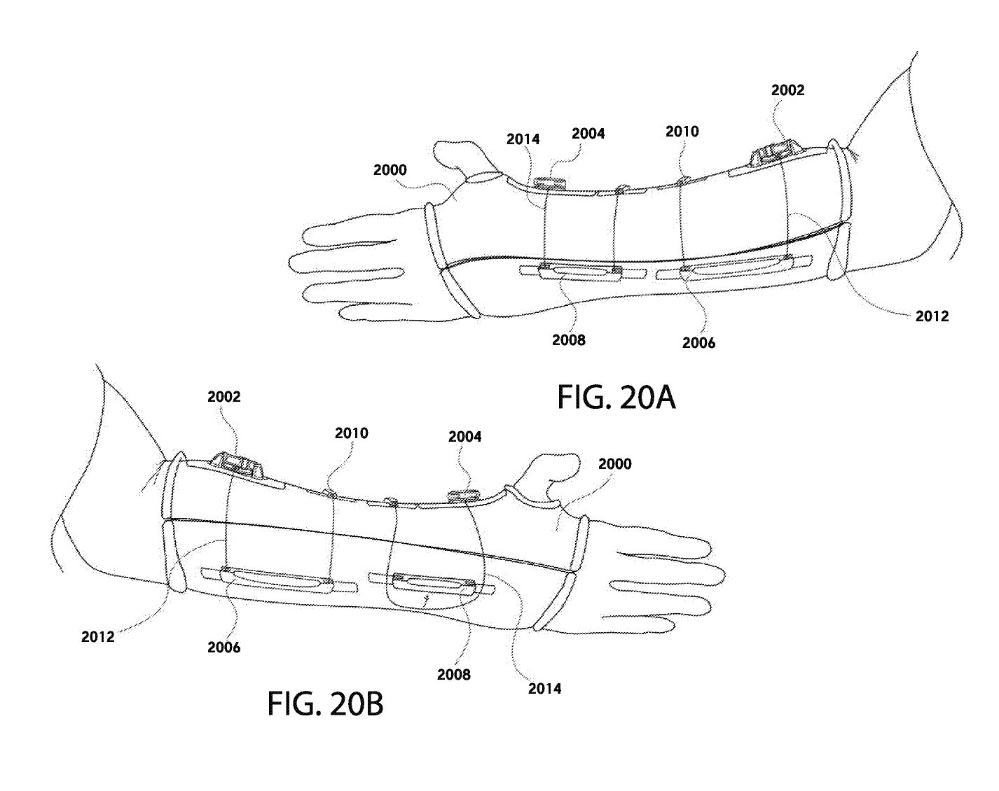

FIGS. 19-20B show tensioning systems on bivalved casts or braces. FIG. 19 shows a series of reels 1900 attached to flanged portions 1902 positioned along a length of a brace 1904. A series of guides 1906 attached to flanged portions 1908 are positioned across a slit 1912 in the brace 1904. Each guide 1906 may include an open back 1912 such that a lace 1910 may be inserted to the guide 1906 as indicated by the arrow. Lace 1910 may be tensioned by each reel 1900 and directed by one of the guides 1906 to form a tensioning system over slit 1912. Slit 1912 may be cut in a wavy or other nonlinear pattern as shown to prevent misalignment and slippage along a longitudinal length of the brace 1904. Slit 1912 may be linear in some embodiments.

FIGS. 20A and 20B show an front side and a back side, respectively, of a bivalved brace 2000 corresponding to the front and back of a user's arm. Brace 2000 may include two reels 2002 and 2004 disposed along a top of the brace 2000. Larger open back guides 2006 may be included on both the interior side and the exterior side of brace 2000 along a proximal portion of brace 2000. Smaller open back guides 2008 may be included on both the front side and the back side of brace 2000 along a distal portion of brace 2000. Guides 2010 may be included on a top of the brace 2000. The reel 2002 may tension a lace 2012 that is directed by a combination of guides 2006 and a proximal most guide 2010 to form a tensioning system along a proximal portion of the brace 2000. Reel 2004 may tension a lace 2014 that is directed by a combination of guides 2008 and a distal most guide 2010 to form a tensioning system along a distal portion of the brace 2000.

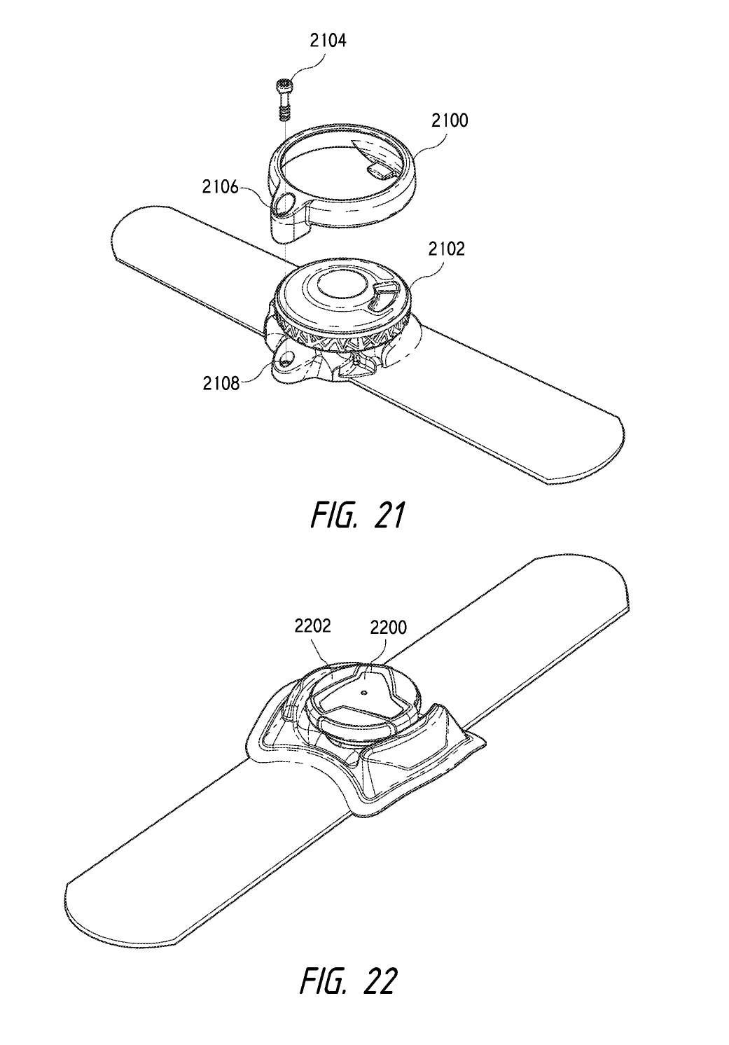

In some embodiments, a physician may want to prevent a patient from adjusting or over-adjusting an amount of tension applied by a reel within a selected range. FIGS. 21 and 22 show embodiments of patient compliance components that control the amount of adjustment a patient may make in the tension applied by the reel. FIG. 21 shows a compliance cap 2100 that may be secured to a reel 2102 using a fastener 2104. In some embodiments, the fastener 2104 may be a screw that fits through an aperture 2106 on cap 2100 and interfaces with a threaded portion 2108 of reel 2102. A physician may tension a lace using reel 2102 and then secure cap 2100 on reel 2102 to limit an amount of adjustment the patient may make. In some embodiments, cap 2100 eliminates the ability of a patient to modify tension. In other embodiments, cap 2100 permits the modification of tension within a predetermined range as set by the physician. A physician may tension the lace an initial amount prescribed to treat a certain type of injury. FIG. 22 shows a shield 2200 that locks or otherwise prevents a reel 2202 from being adjusted by a patient. Embodiments of patient compliance components are described in U.S. patent application Ser. No. 62/170,614, filed Jun. 3, 2015, entitled “Devices and Methods for Facilitating Patient Compliance With Therapeutic Procedures,” the entire contents of which are hereby incorporated by reference. In some embodiments, a lace tender may be included on a cast. A tender is a component that may capture excess lace length such that when the lace is not under tension, the lace may be maintained in a controlled positioned, and excess lace stored.

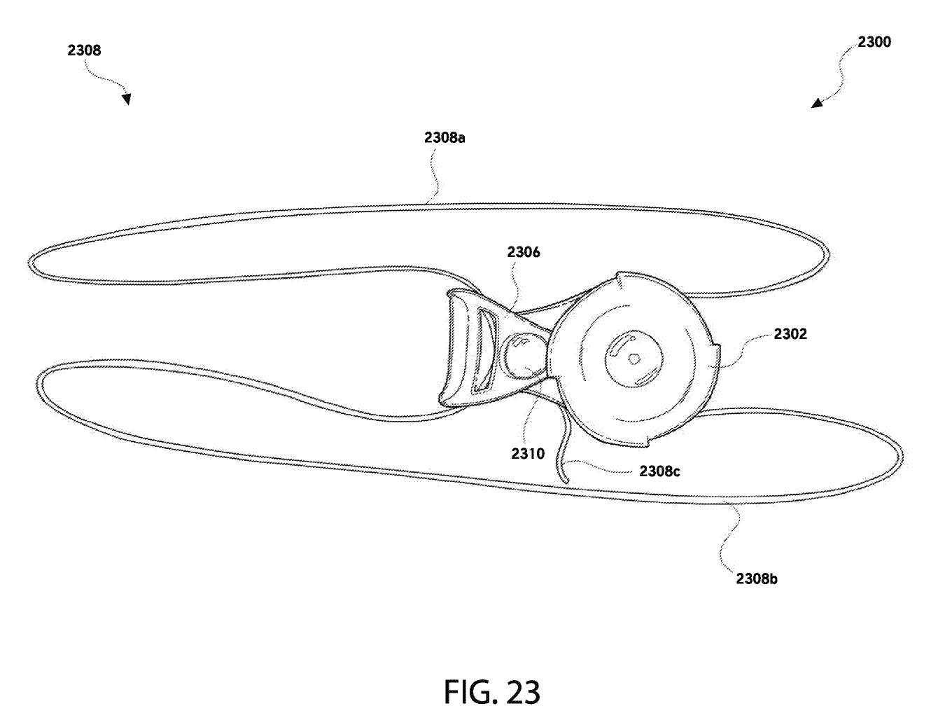

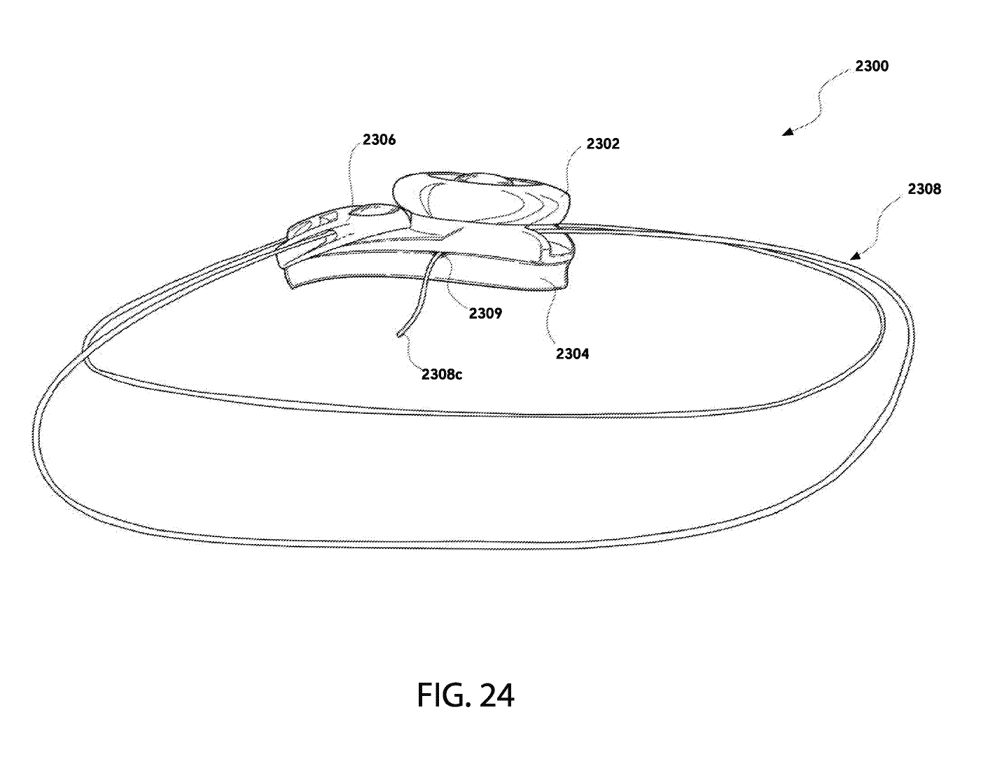

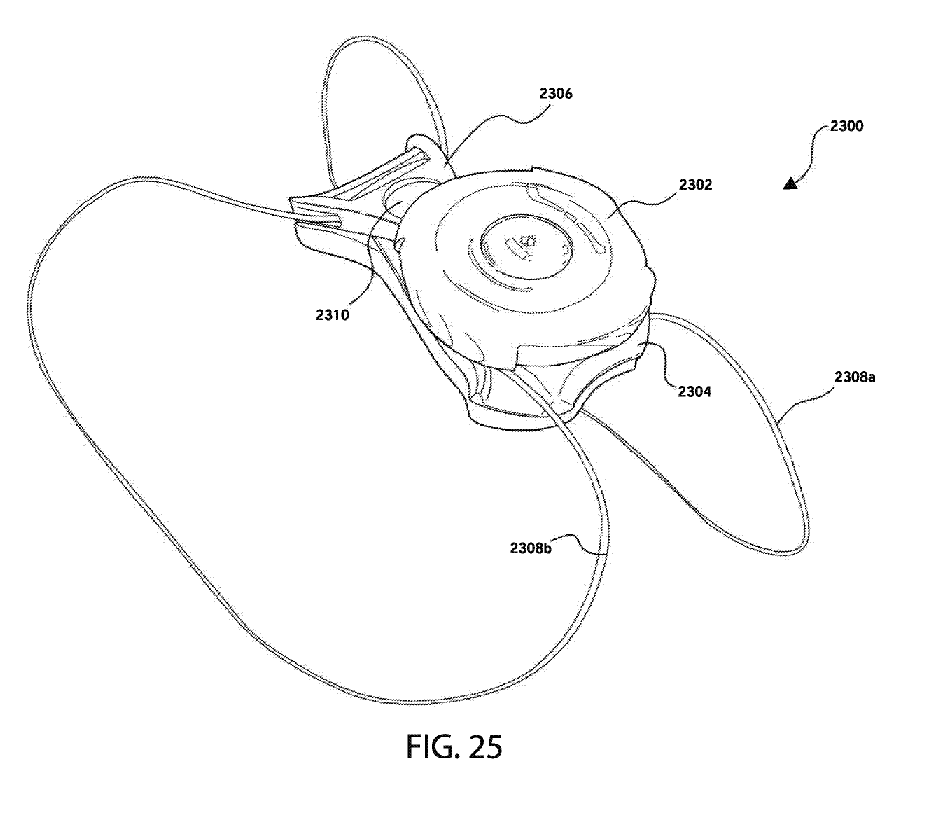

Another exemplary embodiment of a tightening mechanism for use in a bivalve cast is illustrated in FIGS. 23-25. Tightening mechanism 2300 includes a reel assembly having a knob or reel 2302 (hereinafter knob 2302) that is attached to a housing 2304.

The knob 2302 is operable to cause tensioning of a tension member 2308 (hereinafter lace 2308), which in the illustrated embodiment is a lace or cord, although in other embodiments may be a strap or other tensionable component. The knob 2302 may be operated upon a user grasping and rotating the knob 2302. The lace 2308 is attached to a spool (not shown) that is positioned within the housing 2304. Rotation of the knob 2302 causes the spool to rotate within the housing 2304, which winds the lace 2308 around a central post or annular channel. As provided above, exemplary embodiments of reel assemblies that may be used to tension the lace 2308 are further described in the '657 patent, the '521 application, and the '024 application, which are incorporated by reference herein. As described in those applications, the reel assembly may be configured so that rotation of the knob in a first direction effects incremental tightening of the lace and so that rotation of the knob in a second and opposite direction effects incremental loosening of the lace. In some instances, the reel assembly may not operable to allow the spool to freely rotate within the interior region of the housing and thereby fully release tension on the lace. In other instances, the reel assembly may be operable so that the spool is freely rotatable within the interior region of the housing to thereby full release tension on the lace. For example, the knob of the reel assembly may be pulled or lifted axially upward or may be turned in a counter or loosening direction to decouple the spool from the knob or another member that prevents counter rotation of the spool.

In some embodiments, the reel assembly may be configured with a clutch mechanism that limits the tension that may be applied to the lace. An exemplary embodiment of a reel with a clutch mechanism is described in the '690 application, which is incorporated by reference herein.

In other embodiments, the reel assembly may be configured so as limit the user's ability to tension or loosen the lace, thereby ensuring a patient's compliance with a prescribed usage of the cast or brace. Exemplary embodiment of such reel assemblies are described in the '614 application, which is incorporated by reference herein. An exemplary embodiment of a non-reel based tightening mechanism that may be used with the embodiments described herein is further described in U.S. patent application Ser. No. 14/166,799, filed Jan. 28, 2014, and titled “Lace Fixation Assembly and System,” the entire disclosure of which is incorporated by reference herein.

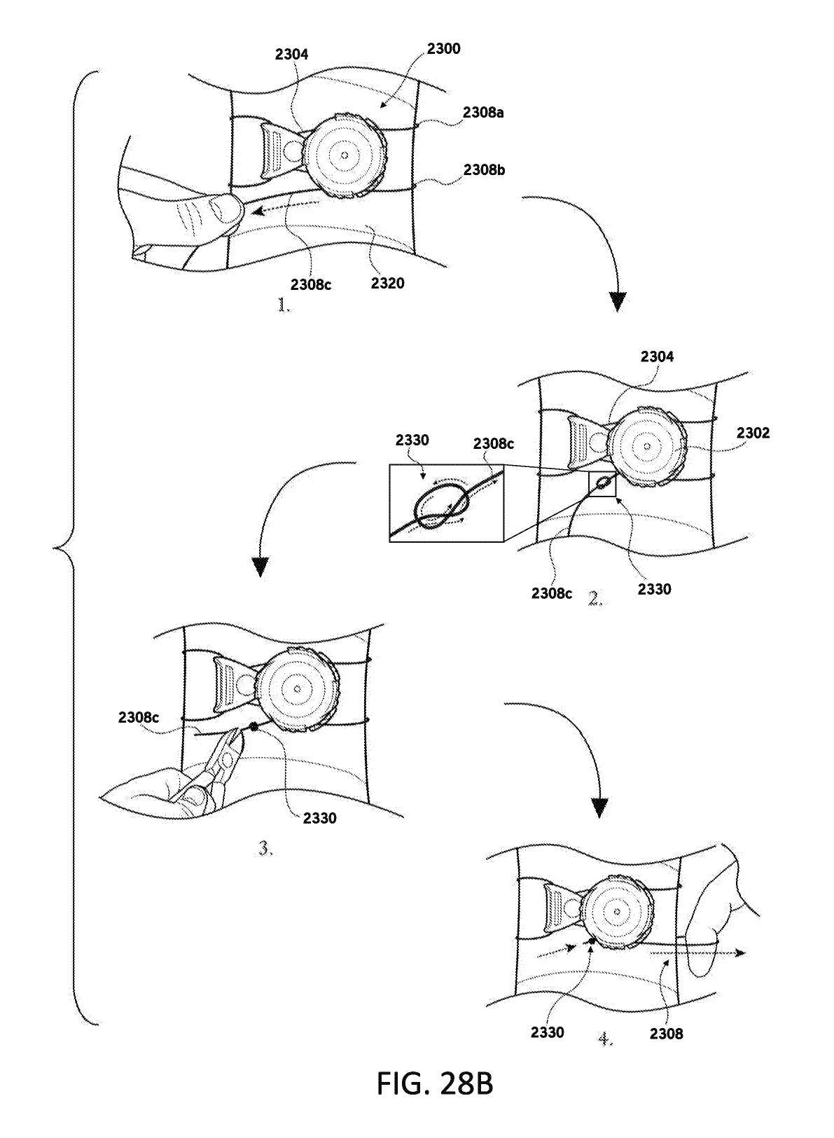

As illustrated in FIGS. 23-25, the lace 2308 may be arranged to form a first loop 2308a and a second loop 2308b. The first and second loops, 2308a and 2308b, may be formed by attaching a proximal end of the lace 2308 to the spool positioned within the housing 2304, forming the loops and routing a midsection of the lace 2308 through a guide 2306 that is positioned or formed on an opposite end of the housing 2304, and attaching a distal end the lace 2308 to the housing 2304 or to the spool positioned within the housing 2304. In some embodiments, a distal end 2308c of the lace 2308 may pass through a lumen or channel of the housing 2304 and exit a port 2309 so that the distal end is uncoupled or unattached with the housing 2304 or any other component of the tightening mechanism. As illustrated in FIG. 28B, in such embodiments the distal end 2308c of the lace 2308 may be used to perform an initial gross or macro adjustment of the lace 2308 that is fit about a cast and tensioned by the tightening mechanism 2300.

Upon operation of the reel assembly in a manner that causes tensioning of the lace 2308 (e.g., rotation of knob 2302), a diameter of the first and second loops of lace, 2308a and 2308b, is reduced so that the two loops of lace, 2308a and 2308b, constrict about an object positioned within the loops, such as a bivalve cast as described in detail herein below. In contrast, upon operation of the reel assembly in a manner that causes loosening of the lace 2308 (e.g., counter-rotation of the knob 2302), the diameter of the first and second loops of lace, 2308a and 2308b, is enlarged so that the two loops of lace, 2308a and 2308b, loosen about the object positioned within the loops, such as a bivalve cast. Although FIGS. 23-25 illustrate the lace 2308 forming two loops, in other embodiments the lace 2308 may form a single loop or three or more loops as desired. In some embodiments, the lace may be coupled with the object (e.g., bivalve cast) so that operation of the reel assembly in a loosening direction causes the object to open.

As further illustrated in FIGS. 23-25, in some embodiments the guide 2306 may be attached to the housing 2304 via a fastening mechanism 2310, such as a rivet or screw. As described herein, in other embodiments the guide 2306 may be integrally formed with the housing 2304 or coupled therewith via other means, such as stitching, adhesive, heat, or RF bonding. In any event, the guide 2306 typically includes a lumen or channel within which the lace 2308 slides. The lumen or channel of the guide 2306 is commonly made of a low friction material to reduce tension loss in the lace due to friction and/or to reduce unnecessary wear on the lace 2308. In the embodiments where the lace 2308 forms a single loop, the tightening mechanism 2300 may not include the guide 2306. Rather, a distal end of the lace 2308 may be attached or otherwise coupled with the distal end of the housing 2304. Further, although FIGS. 23-25 illustrate a reel assembly being used to tension the lace, it should be realized that other mechanisms could be used to tension the lace, such as pull cords, motorized devices, and the like.

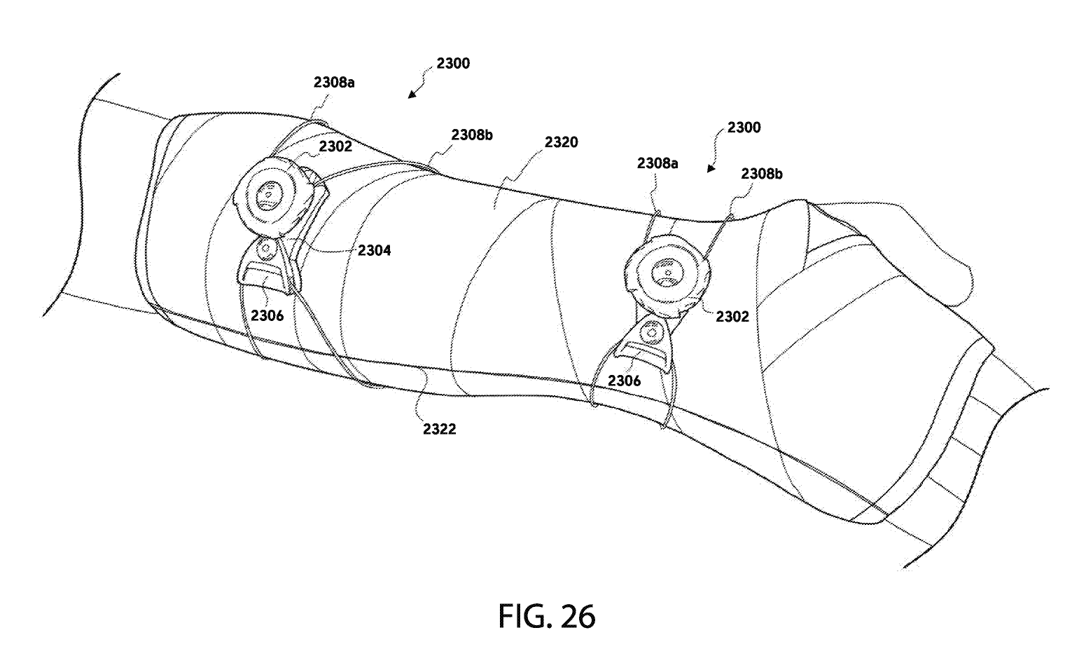

Referring now to FIG. 26, illustrated is a tightening mechanism 2300 of FIGS. 23-25 positioned about a bivalve cast 2320. Specifically, two tightening mechanisms 2300 are positioned on the bivalve cast 2320 towards opposite ends thereof. The bivalve cast 2320 includes a longitudinal cut 2322 that divides the bivalve cast 2320 and allows for pressure relief of a limb positioned within the bivalve cast, such as the illustrated forearm. Operation of the knob 2302 causes tensioning of the lace 2308, which constricts the first loop of lace 2308a and the second loop of lace 2308b about the bivalve cast 2320, thereby causing the bivalve cast 2320 to constrict or tighten about the limb. A wider longitudinal cut 2322 in the cast 2320 allows for a greater degree of pressure to be applied to the limb, so as to compensate for atrophy of the limb and/or for any other reason. In some embodiments, the knob 2302 may be operated in a second manner (i.e., counter-rotation of the knob) to effect loosening of the lace 2308, which opens the first and second loops of lace, 2308a and 2308b, thereby causing the bivalve cast 2320 to open or loosen about the limb.

In embodiments that allow incremental loosening of the lace 2308, the tightness of the bivalve cast 2320 about the limb may be quickly and easily adjusted simply by operating the knob 2302 until a desired tightness is achieved. For example, the knob 2302 may be counter-rotated by a few angular degrees to relieve an overly-tight pressure on the limb. The process does not involve a complete loosening and subsequent retightening of the bivalve cast as the use of conventional gauze requires.

It should be realized that although FIG. 26 illustrates two tightening mechanisms 2300 being used, in other embodiments a single tightening mechanism 2300 or three or more such mechanisms may be used. For example, given the relatively rigid nature of bivalve casts, a single tightening mechanism 2300 may be sufficient to close and tighten the bivalve cast 2320 about a limb. In other embodiments, a slight increase in pressure may be desired in one or more specific locations of the bivalve cast 2320. In such instances multiple tightening mechanisms 2300 may be positioned about the bivalve cast 2320 at the specific locations to provide the desired pressure in the specific location or locations.

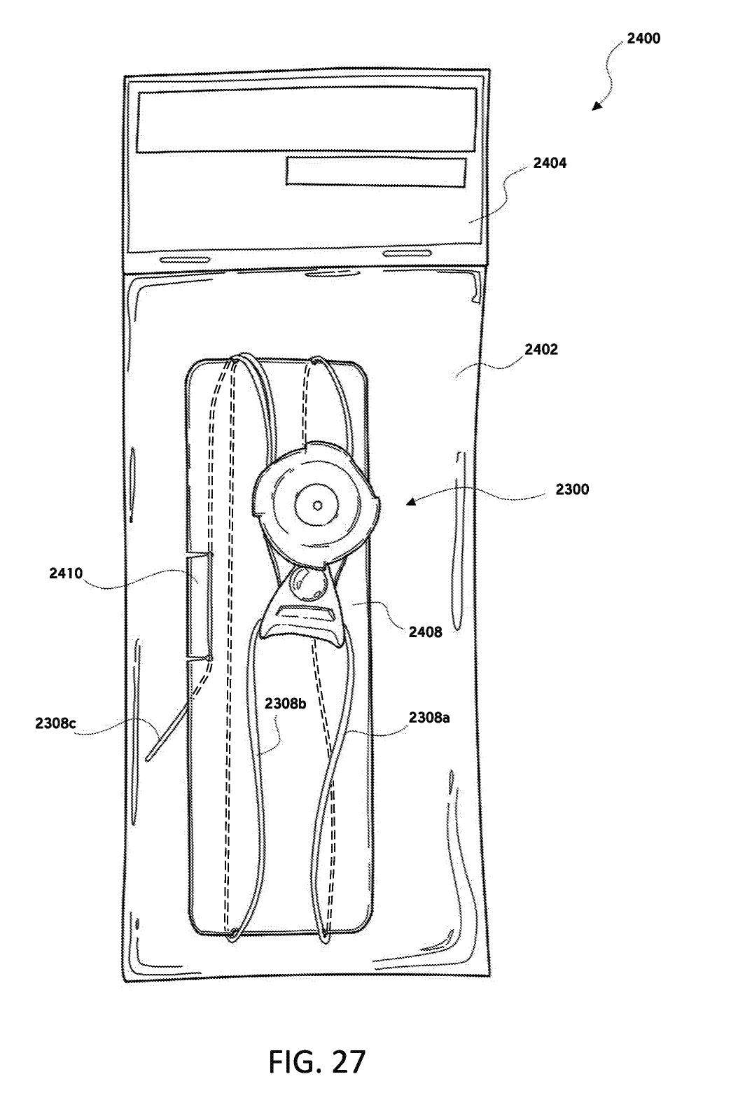

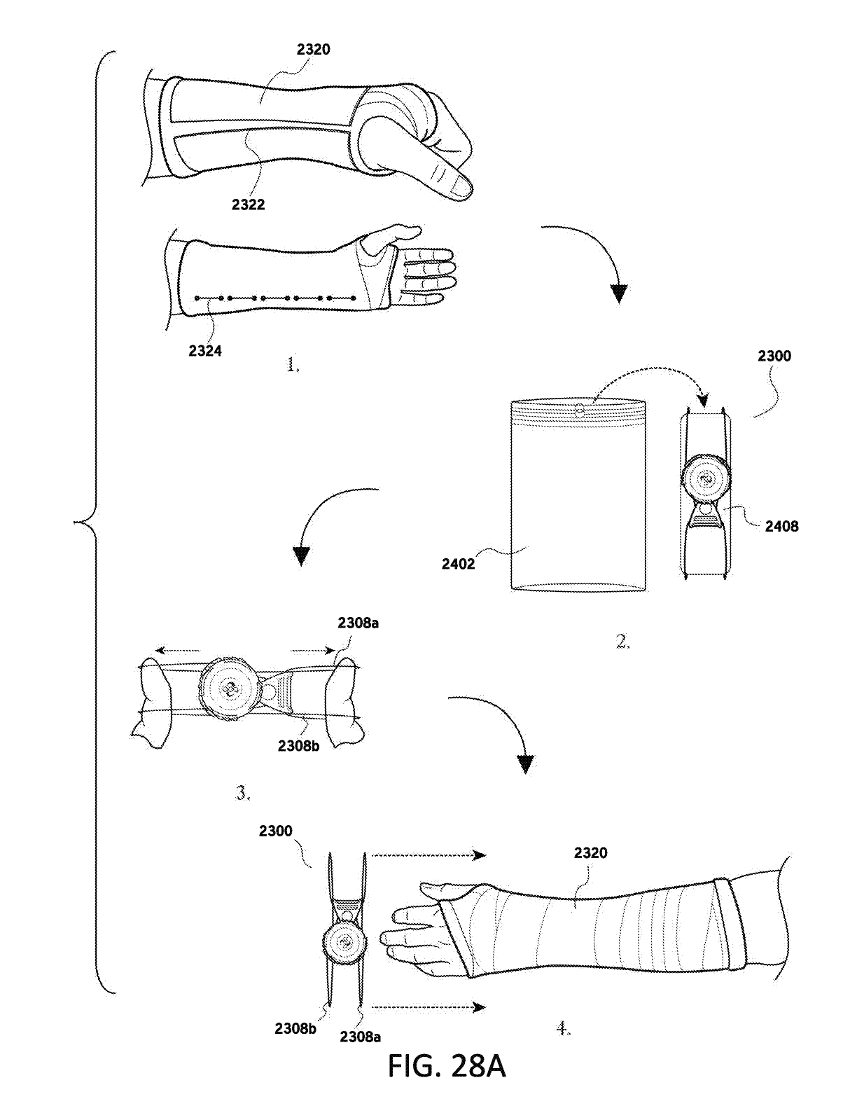

Referring now to FIG. 27, illustrated is a packaging 2400 of the tightening mechanism 2300. The packaging 2400 includes a carrying portion 2402 that encloses and houses the tightening mechanism 2300, and includes a tag 2404 having indicia or other means of identifying the tightening mechanism product. The carrying portion 2402 may be a plastic or other similar type sleeve within which the tightening mechanism 2300 is disposed. The packaging 2400 may also include a component 2408 that couples with the lace 2308 to aid in preventing tangling of the lace within the sleeve 2402. Specifically, the first and second loops of lace, 2308a and 2308b, may be wrapped or disposed around the body of the component 2408 so that the component 2408 maintains the loops of lace, 2308a and 2308b, in an open configuration and prevents the loops of the lace from tangling or kinking within the sleeve 2402. In some embodiments, a distal end 2308c of the lace may be coupled with a tab 2410 or cut out portion of the component 2408. Coupling of the distal end 2308c of the lace with the tab 2410 may prevent the distal end 2308c of the lace from being pulled through and/or otherwise detaching from the housing 2308 of the tightening mechanism 2300. The distal end of the lace 2308c may be relatively free or unattached from the housing 2304 or another component of the tightening mechanism 2300 to allow the loops of lace, 2308a and 2308b, to be adjusted as illustrated and further described in relation to FIG. 28B.

FIGS. 28A and 28B illustrate a method of using the tightening mechanism 2300 on a bivalve cast 2320. Specifically, a bivalve cast 2320 may be fit about a patient, such as a forearm as illustrated in FIG. 28A. The bivalve cast 2320 includes one or more longitudinal cuts 2322 that allow pressure on the limb to be relieved and/or that aid in positioning the bivalve cast 2320 on the limb. The bivalve cast 2320 may also include one or more slits 2324 that are positioned opposite the longitudinal cut 2322 that aid in opening the bivalve cast 2320 and/or in positioning the bivalve cast 2320 about the users limb. For example, the slits 2324 function similar to hinges to enable the bivalve cast 2320 to be opened about the longitudinal cut 2322.

After the bivalve cast 2320 is positioned about the users limb, the tightening mechanism 2300 may be removed from its packaging 2400, such as by removing the tightening mechanism 2300 from a plastic sleeve 2402. The tightening mechanism 2300 may also be removed from a component 2408 about which is positioned, such as by loosening the first and/or second lace loops, 2308a and 2308b. A diameter of the lace loops, 2308a and 2308b, may be widened or enlarged to accommodate larger bivalve casts 2320 and/or to allow the lace loops to be easily positioned over the bivalve cast 2320. The lace loops, 2308a and 2308b, may be widened by a user positioning their fingers within the lace loops and pulling opposing portions of the lace loops in opposite directions. The bivalve cast 2320 may then be inserted within the lace loops, 2308a and 2308b, so that the tightening mechanism 2300 and lace 2308 is positioned atop the bivalve cast. The tightening mechanism 2300 may then be operated as described above to constrict the lace loops, 2308a and 2308b, about the bivalve cast 2320.

In some instances it may be desirable to perform a gross or macro adjustment of the lace loops about the bivalve cast prior to operating the tightening mechanism 2300. Performing the gross or macro adjustment of the lace loops may reduce the amount of lace that needs to be wound or stored within the housing 2304 and around the tightening mechanism's spool. The reduced lace storage and/or lace windings may allow the size of the reel assembly to be greatly reduced, resulting in a significantly smaller mechanism being positioned atop the bivalve cast. The gross or macro adjustment of the lace loops is typically performed when the spool is essentially free of lace, or in other words when the lace is not wound about the spool's central channel or post. This ensures that the cast may be accurately fit to the limb and that the reel assembly cannot be operated to overly loosen the lace.

FIG. 28B illustrates an embodiment of the tightening mechanism 2300 in which the tightening mechanism 2300 is configured to allow gross or macro adjustment of the lace 2308 prior to operation of the tightening mechanism 2300. Specifically, a distal end 2308c of the lace 2308 is free or otherwise uncoupled from or unattached to the housing 2304. This may be achieved by positioning a distal portion of the lace 2308 through a lumen (not shown) of the housing 2304. The distal portion of the lace 2308 is slidingly positioned within the housing's lumen so that the lace's distal portion is able to slide within the lumen and relative to the housing 2304. Sliding of the lace's distal portion enables the first and second lace loops, 2308a and 2308b, to be enlarged or widened as a user pulls on the opposing portions of the lace loops as shown in FIG. 28A. As shown in illustration 1 of FIG. 28B, after the tightening mechanism 2300 is positioned about the bivalve cast 2320, the distal end 2308c of the lace may be grasped and pulled, which causes the distal portion of the lace 2308 to slide within the housing's lumen and thereby constricts the lace loops, 2308a and 2308b, about the bivalve cast 2320. In this manner, a gross or macro adjustment of the lace loops, 2308a and 2308b, is achieved without, and prior to, the operation of the tightening mechanism 2300. The lace loops, 2308a and 2308b, are also adjusted to accommodate for the specific size of the bivalve cast 2320.

As shown in illustration 2 of FIG. 28B, a knot 2330 is then be tied in the distal end 2308c of the lace 2308. A crimp or other securement component may alternatively, or additionally, be coupled or attached to the lace 2308 instead or, or in addition, to the use of the knot 2330. As shown in illustration 3, the distal end 2308c of the lace 2308 that is distal to or beyond knot 2330 may be cut and removed. As shown in illustration 4, the knot 2330, crimp, or other securement component, engages with the port 2309 of the housing 2304 upon tensioning of the lace 2308 to prevent the distal portion of the lace 2308 from being pulled through the housing's lumen and thereby loosening the lace loops 2308a and 2308b. In some embodiments, the port 2309 may be recessed into the housing 2304 so that the distal end 2308c of the lace is not visible. The lace 2308 may be tensioned via the tightening mechanism 2300 or via the user pulling on a proximal portion of the lace 2308. Using the process illustrated in FIG. 28B, the portion of the lace 2308 that is tensioned via the tightening mechanism 2300 is reduced prior to operation of the tightening mechanism 2300. Because the tensioned portion of the lace is reduced prior to operation of the tightening mechanism 2300, the tightening mechanism 2300 does not need to be configured to store the excess lace that is not used in tightening the bivalve cast 2320. As such, smaller sized tightening mechanisms may be used.

Using the process illustrated in FIG. 28B also aids in ensuring that the patient complies with a proper usage and fit of the cast about the limb. For example, since usable lace length is reduced in performing the gross or macro adjustment process, the patient is not able to loosen the lace to a point that allows the cast to be removed from the limb. Thus, even though the cast may be loosened to provide comfort and/or to accommodate swelling of the limb, the cast will remain fit and secured about the limb.

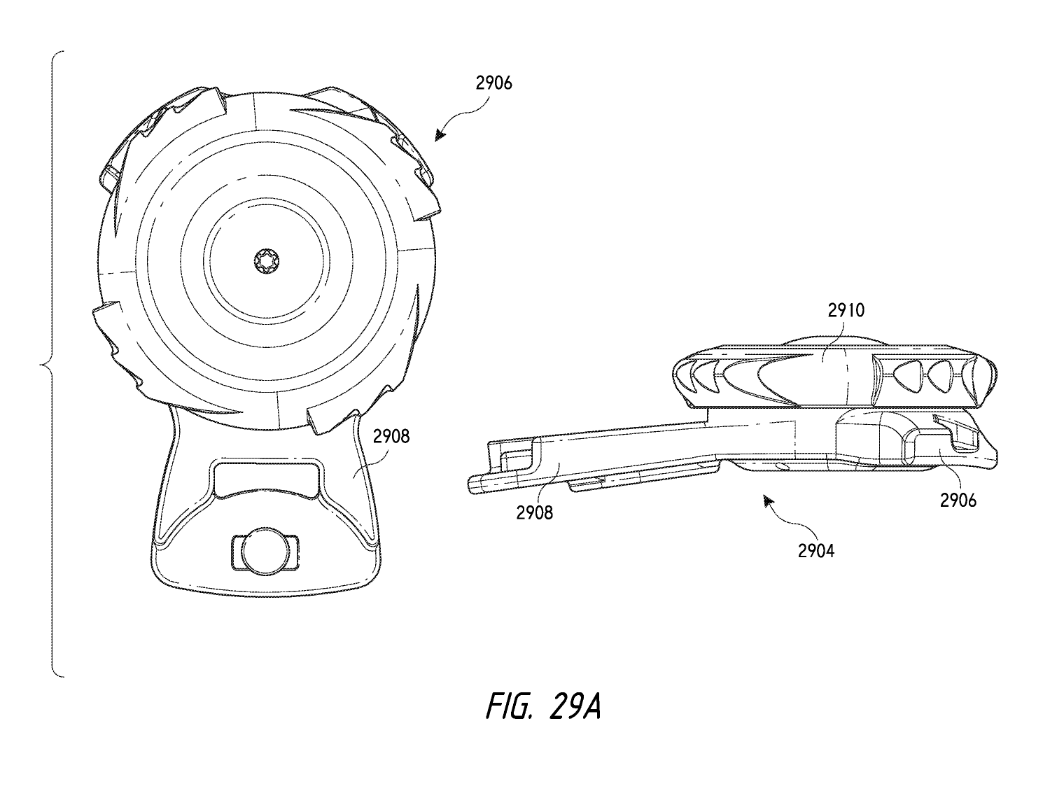

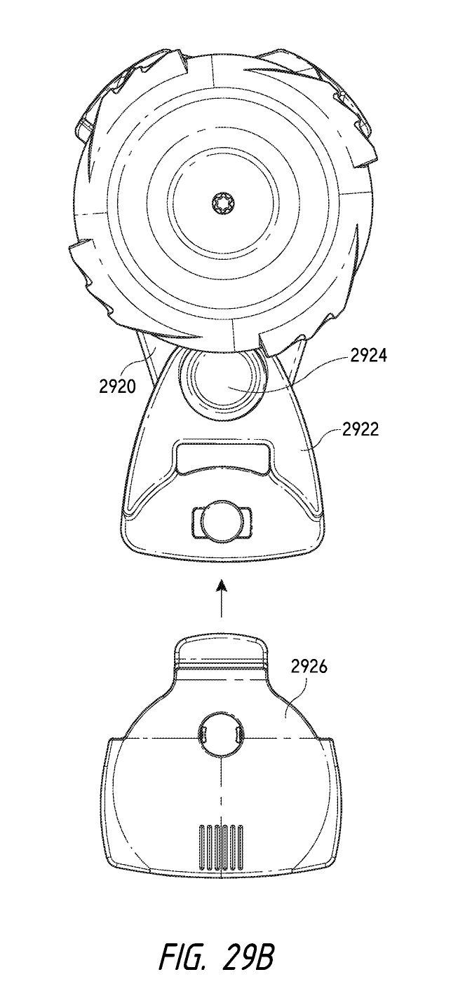

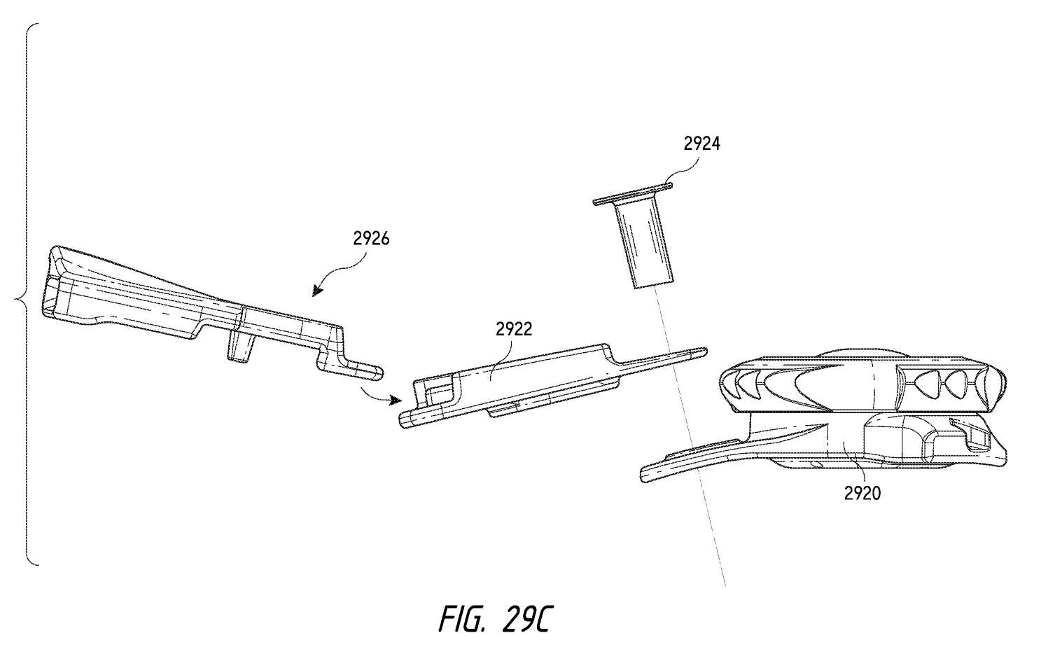

FIGS. 29A-C illustrate other embodiments of housings for the tightening mechanism. Specifically, FIG. 29A illustrates an integrated or single component housing 2904. The housing 2904 includes a proximal end 2906 that is coupled with the tightening mechanism's knob 2910, and includes a distal end 2908. The distal end 2908 may function as a lace guide similar guide 2306 and/or include a lumen or channel (not shown) that guides or routes the lace. In another embodiment, the distal end 2908 may include a female coupling component that is configured to removably couple with a corresponding male component. Exemplary male and female coupling components that may be employed for the distal end 2908 of housing 2904, or for the other embodiments described herein, are further described in U.S. patent application Ser. No. 14/071,435, the entire contents of which are incorporated by reference herein.

FIGS. 29B and 29C illustrate another embodiment of a housing 2920. A proximal end of the housing 2920 is coupled with the tightening mechanism's knob while a distal end of the housing 2920 is coupleable with a female coupling component 2922, such as those described in the '435 patent application. The female coupling component 2922 may be coupled with the distal end of the housing 2920 via a rivet 2924, screw, mechanical fastener, adhesive bond, sonic or other weld, heat bond, and the like. A male component 2926, such as those described in the '435 patent application, may be removably coupled with the female component 2922. An advantage of using the male and female coupling components is the ability to position the lace around and about the bivalve cast simply by coupling the male and female components, 2926 and 2922, together. In some embodiments, the female component 2922 may not be coupled with the housing, but rather coupled directly to the cast. The male component 2926 may be attached to the female component 2922 that is attached directly to the cast. In yet other embodiments, the male component 2926 may be attached to the housing or directly to the cast and the female component 2922 may be removably attached to the male component 2926.

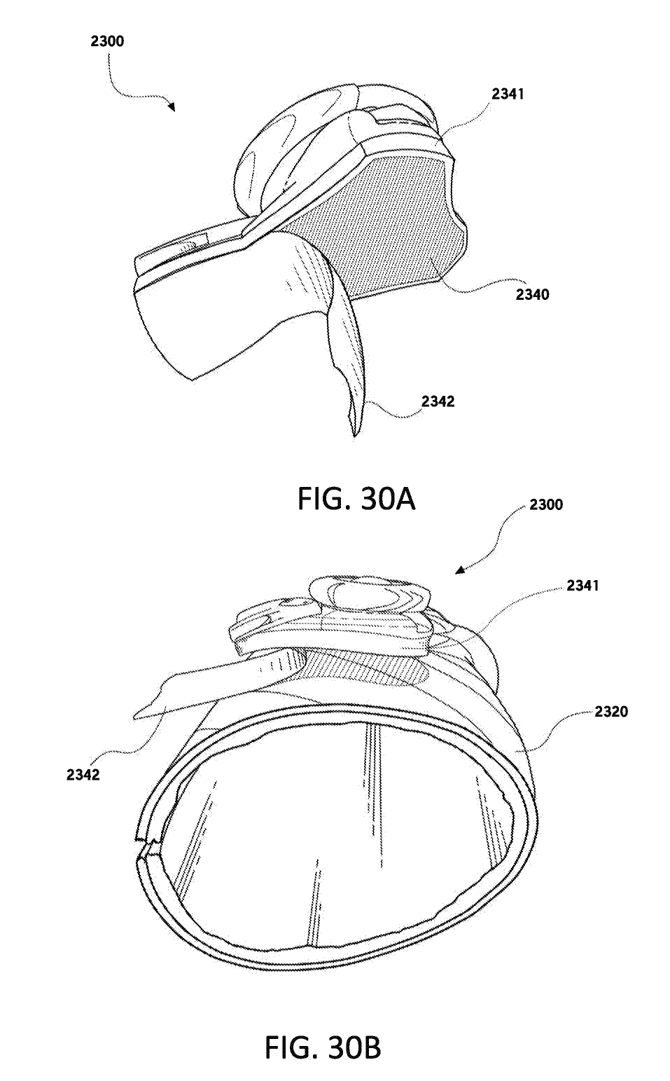

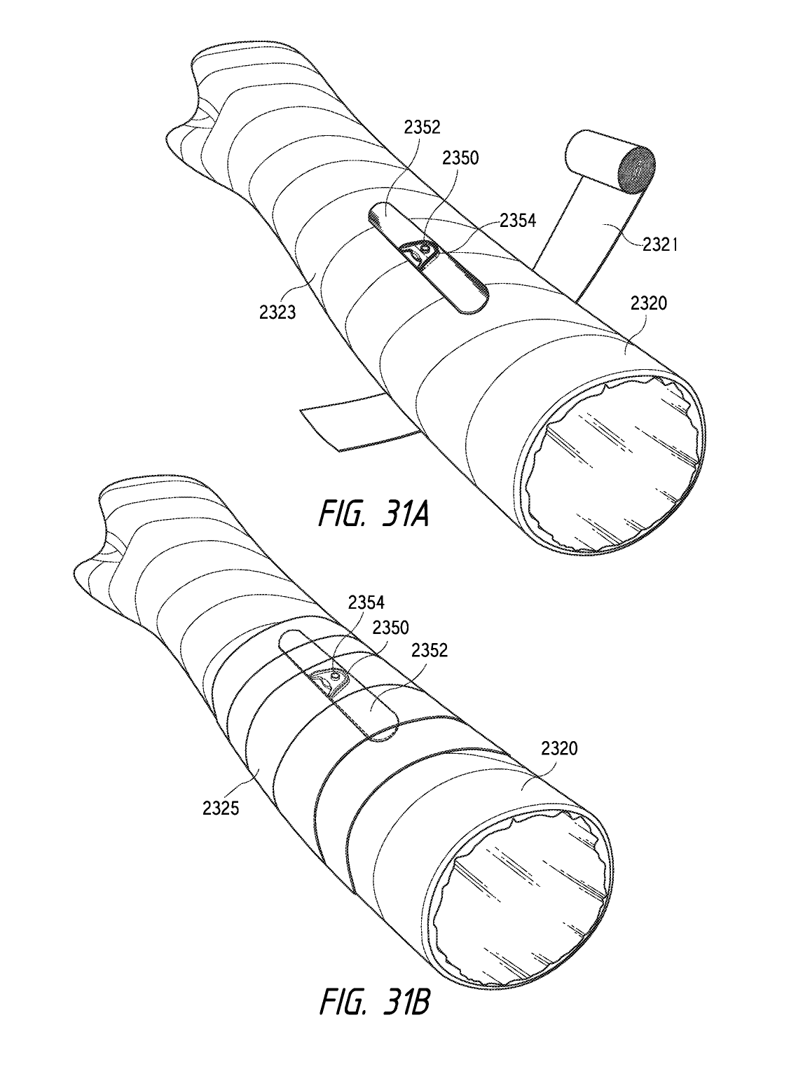

FIGS. 30A-B illustrates an embodiment of coupling the tightening mechanism 2300 with the body of the bivalve cast 2320. Specifically, a bottom surface of the tightening mechanism 2300 may include an adhesive material 2340 that adhesively bonds the tightening mechanism 2300 with the body of the bivalve cast 2320. Exemplary adhesive materials include: cyanoacrylates, such as those sold under the trade names Super Glue° and Krazy® Glue; preformed adhesive materials, such as those sold under the trade name Glue Dots®; and the like. If used with the foam layer 2341 described below, the cyanoacrylates or adhesive materials bond very well with the cast's fiberglass material. A releasable liner 2342, such as a plastic strip of material, may be positioned over the adhesive material 2340 to protect the adhesive material 2340 from degradation and prevent incidental bonding of the tightening mechanism 2300 with nearby objects. In coupling the tightening mechanism 2300 with the bivalve cast 2320, the tightening mechanism may be moved longitudinally about the cast 2320 to a desired location and then the releasable liner 2342 removed. The tightening mechanism may then be pressed downward onto the outer surface of the cast body to adhere the tightening mechanism 2300 to the cast 2320. Because the tightening mechanism 2300 is adhered to the cast body, subsequent tensioning of the lace 2308 will not cause the tightening mechanism 2300 to migrate or move about the cast 2320. In some embodiments, the adhesive material may be sufficient to couple the tightening mechanism 2300 to the cast 2320 to prevent movement of the tightening mechanism during tensioning of the lace, but may not permanently adhere the tightening mechanism 2300 to the cast 2320. In such embodiments, the user may reposition the tightening mechanism 2300 about the cast 2320 as desired. One method of repositioning the tightening mechanism 2300 may include using a tightening mechanism 2300 with a high coefficient of friction on its bottom surface. The high coefficient of friction may enable the tightening mechanism 2300 to grip the cast without permanently bonding thereto.

In some embodiments, the tightening mechanism 2300 may include a foam layer 2341 positioned on a bottom surface of the tightening mechanism 2300. The foam layer 2341 may aid the tightening mechanism 2300 in conforming to different sized casts. For example, the foam layer 2341 may compress against the cast's outer surface and thereby conform to the shape and size of the cast 2300. The tightening mechanism 2300 may be slightly arcuated or curved to further aid in conforming the tightening mechanism 2300 to the shape and size of the cast. In this manner, the tightening mechanism 2300 conforms to the contour of the cast 2320, which prevents the creation of point loads/pressure and/or other non-conformance issues.

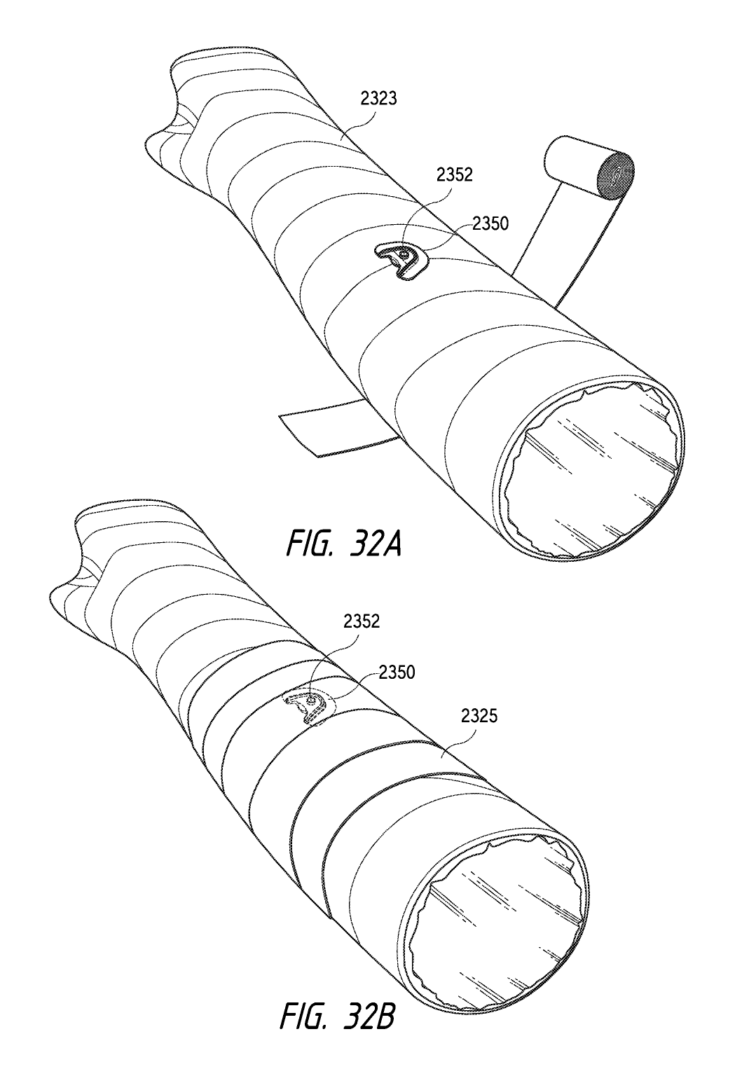

FIGS. 31A-34 illustrate another embodiment of coupling a tightening mechanism 2300 with a bivalve cast 2320. Specifically, as shown in FIGS. 31A-32B, a coupling component 2350 may be integrally formed with the bivalve cast 2320. The coupling component 2350 may be integrally formed with the cast 2320 by positioning the component 2350 under the outermost gauze layer or layers of the cast 2320. For example, the cast's gauze 2321 may be wrapped one or more times to form an inner layer 2323 and then the coupling component 2350 may be positioned atop the inner layer 2323 of the cast 2320. The gauze 2321 may then be wrapped one or more times about the coupling component 2350 and/or about the remainder of the cast 2320 to form an outer layer 2325 atop the component 2350 and/or remainder of the cast 2320. Polyurethane or another casting material may then be applied to the inner and/or outer layers of the cast 2320 to harden the inner layer 2323 and/or outer layer 2325 with the coupling component 2350 positioned between the inner and outer layers.

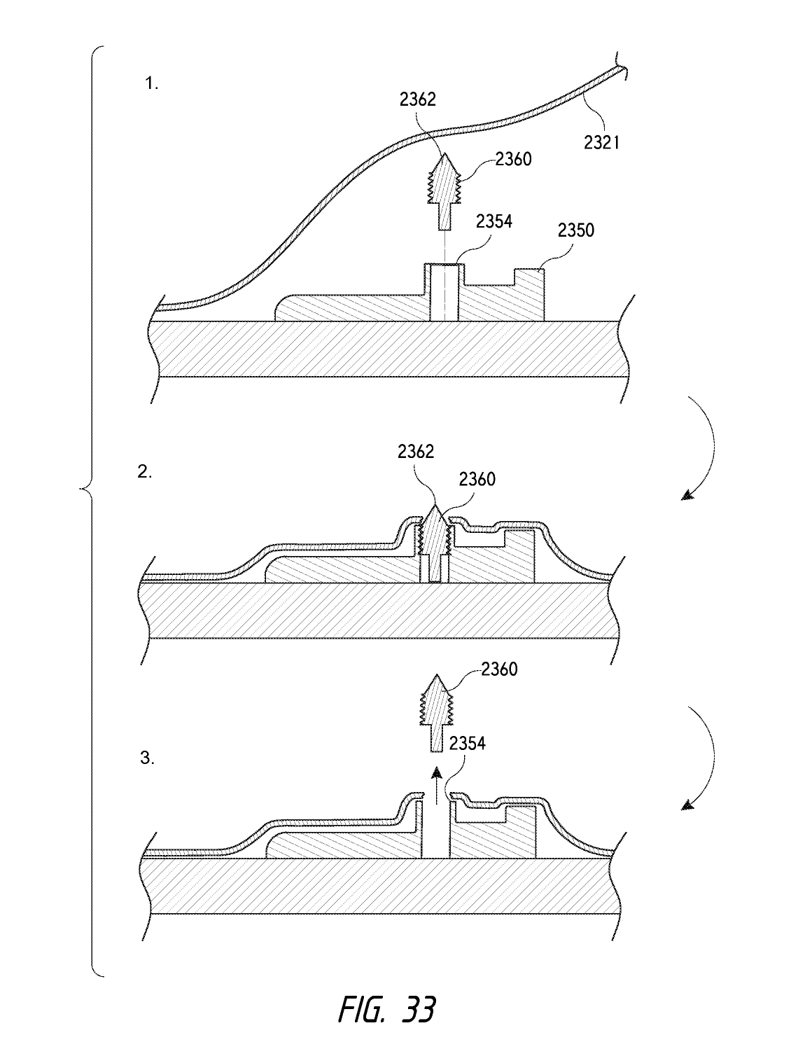

In some embodiments, the coupling component 2350 may include flanges or wings 2352 that may aid in stabilizing the components 2350. In other embodiments, the coupling component 2350 may not include such additional features. In either embodiment, the coupling component 2350 includes a mounting aperture 2354 that enables the coupling component to mount with the tightening mechanism's housing. As shown in FIG. 33, an extractable component 2360 may be inserted within the mounting aperture 2354. The extractable component 2360 may aid in cutting or piercing the hardened gauze material positioned atop the mounting aperture 2354 to expose the mounting aperture for subsequent coupling with the tightening mechanism's housing. The extractable component 2360 may be threaded into the mounting aperture 2354 prior to wrapping the gauze 2321 over the coupling component 2350 and forming the hardened outer layer 2325. The extractable component 2360 may have a sharpened distal tip 2362 that pierces through the gauze material 2321 as the gauze 2321 is wrapped over the coupling component 2350. Upon drying and hardening of the cast's outer layer 2325, the extractable component 2360 may be removed from the mounting aperture 2354 thereby exposing the mounting aperture for subsequent coupling with the tightening mechanism's housing. The extractable component 2360 may be removed via the extractable component's threads or by some other means. In some embodiments, the distal end of the extractable component 2360 may be configured to allow a screw driver to remove the extractable component 2360 from the mounting aperture 2354. An additional screw may then be used to secure the tightening mechanism's housing 2304 to the coupling component 2350.

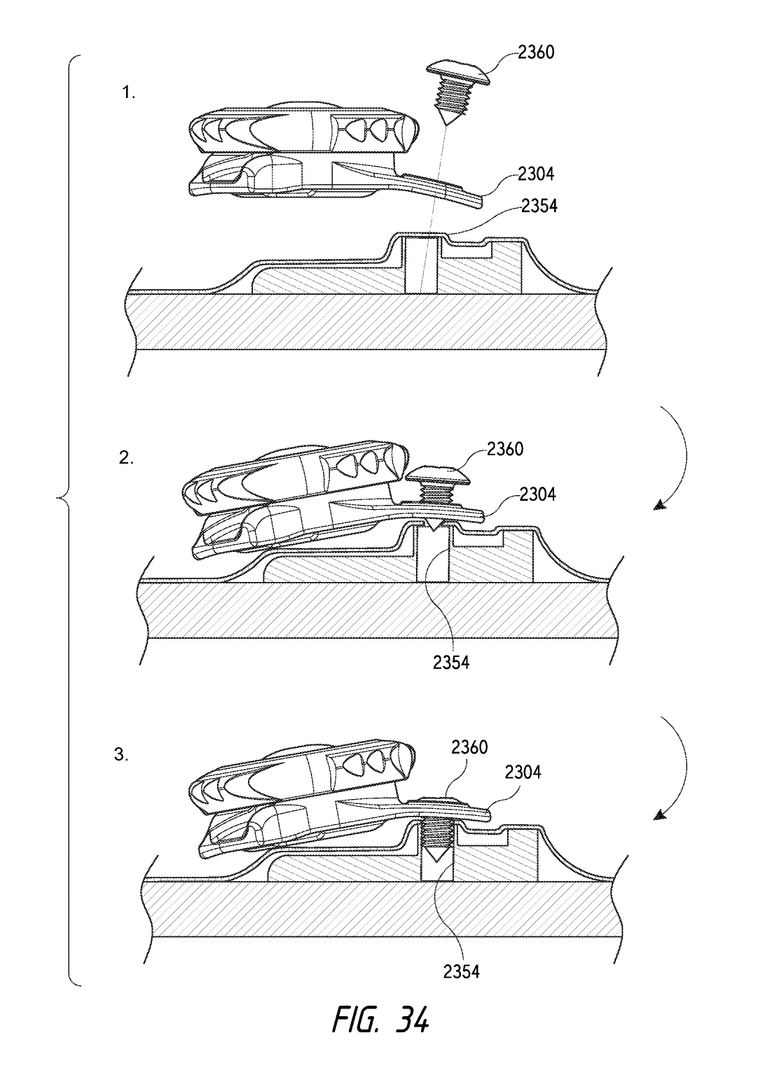

FIG. 34 illustrates an embodiment of coupling the tightening mechanism's housing 2304 to the coupling component 2350. Specifically, a screw 2360 is inserted within an aperture of the housing 2304 and then threaded into mounting aperture 2354 of the coupling component 2350. In some embodiments, the mounting aperture 2354 may be exposed through a hole in the outer layer 2325, which may be formed via the process described in FIG. 33. In other embodiments, the distal tip of the screw 2360 may be pointed and sharp so as to penetrate through the outer layer 2325 as the screw 2360 is inserted into the mounting aperture 2354.

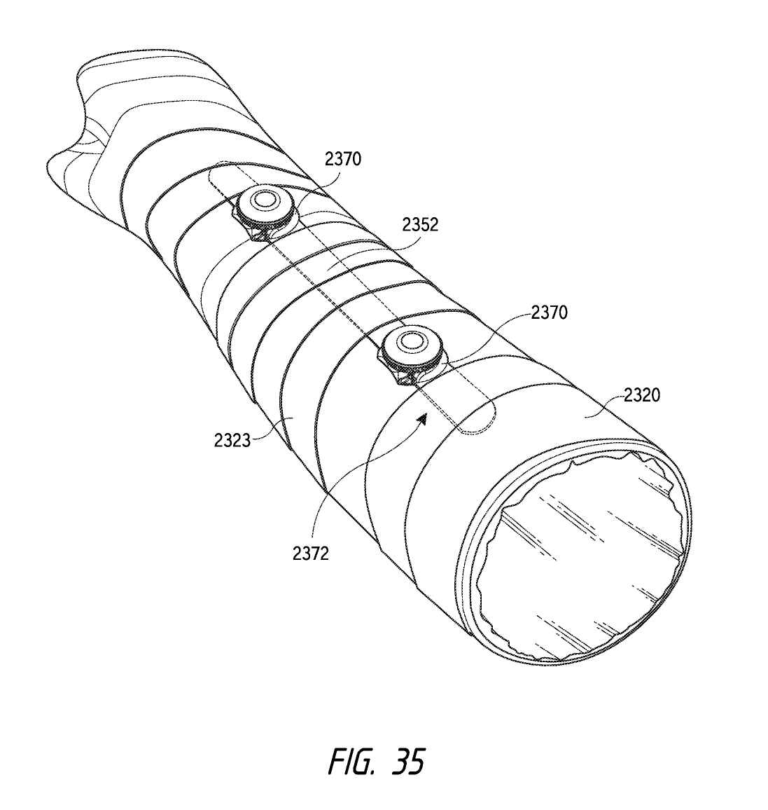

FIG. 35 illustrates an embodiment of a tightening component 2372 that may be integrally formed with the bivalve cast 2320. As described previously, the tightening component 2372 is integrally formed with the cast 2320 by positioning the component 2372 under the outermost gauze layer or layers of the cast 2320 and above an inner layer 2323 of the cast 2320. Polyurethane or another casting material may then be applied to the inner and/or outer layers of the cast 2320 to harden the inner layer and/or outer layer with the tightening component 2372 positioned between the inner and outer layers.

The tightening component 2372 includes flanges or wings 2352 that aid in stabilizing the component 2372 within the cast's inner and outer layers. A pair of tightening mechanisms 2370 are positioned on the flanges 2352 of the tightening component 2372. The tightening mechanisms 2370 may include reel based closure systems, attachment components (e.g., male or female coupling components), or a combination of such components. The use of the two tightening mechanism 2370 may allow the pressure applied to the limb to be varied between the opposing ends of the cast 2320 as desired.

Although the embodiments herein have been described as being used for bivalve casts, it should be realized that a variety of other means may be used to allow the volume of the cast to be changed or adjusted. For example, the cast may include various cuts that allow for the cast's volume to volume, or the cast could include a single or multiple slits or hinges that allow the cast's volume to change. Regardless of the manner used to enable cast volume adjustment, the embodiments described herein may be used with the cast to tighten or loosen the cast about a patient's limb.

Further, it should be realized that the embodiments may be used to close various other devices or articles. For example, any device, article, or component that includes a longitudinal slit or opening, or about which the tightening mechanism may be positioned, may be closed and tightened with the tightening mechanisms described herein. As such, although an exemplary usage of the tightening mechanism is for bivalve casts, various other articles, devices, or components may benefit from usage of the tightening mechanism.

While several embodiments and arrangements of various components are described herein, it should be understood that the various components and/or combination of components described in the various embodiments may be modified, rearranged, changed, adjusted, and the like. For example, the arrangement of components in any of the described embodiments may be adjusted or rearranged and/or the various described components may be employed in any of the embodiments in which they are not currently described or employed. As such, it should be realized that the various embodiments are not limited to the specific arrangement and/or component structures described herein.

In addition, it is to be understood that any workable combination of the features and elements disclosed herein is also considered to be disclosed. Additionally, any time a feature is not discussed with regard in an embodiment in this disclosure, a person of skill in the art is hereby put on notice that some embodiments of the invention may implicitly and specifically exclude such features, thereby providing support for negative claim limitations.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the device” includes reference to one or more devices and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. A device for adjusting a fit of a brace about a limb comprising:
a tightening mechanism comprising:
a housing having an interior region;
a spool disposed within the interior region of the housing, the spool being rotatable within the interior region of the housing; and
a knob that is operably coupled with the spool so that rotation of the knob in a first direction causes the spool to rotate within the interior region of the housing in the first direction; and
a tension member that is coupled with the spool such that rotation of the spool in the first direction causes the tension member to be wound about the spool, wherein the tension member forms a loop that is positionable about the brace to circumferentially surround an outer surface of the brace, wherein winding of the tension member about the spool causes a diameter of the loop to decrease such that after the loop is positioned about over the brace, the tension member constricts about the outer surface of the brace to tighten the brace about the limb, and wherein the tension member is not coupled with any guide that is attached to the brace that guides or directs the tension member about a path.

2. The device of claim 1, further comprising a guide member that is coupled directly with the tightening mechanism to redirect the tension member and thereby define a second loop that is also positionable about the brace to circumferentially surround the outer surface of the brace, wherein the tension member is slidably disposed within a channel of the guide member such that winding of the tension member about the spool causes a diameter of the second loop to decrease simultaneously with the decrease in the diameter of the loop.

3. The device of claim 1, wherein a first end of the tension member is coupled with the spool and a second end of the tension member is adjustably coupled with the housing in a manner that allows the diameter of the loop to be decreased without winding the tension member about the spool.

4. The device of claim 1, wherein a bottom surface of the housing opposite the knob includes an adhesive that is bondable to an outer surface of the brace.

5. The device of claim 1, wherein the tightening mechanism is attachable to a base component that is positioned under an outer layer of the brace.

6. The device of claim 1, wherein the tightening mechanism is configured so that rotation of the knob in the first direction causes the spool to incrementally rotate in the first direction and thereby incrementally tighten the brace about the limb and so that a rotation of the knob in a second direction causes the spool to incrementally rotate in the second direction and thereby incrementally loosen the brace about the limb.

7. The device of claim 6, wherein the tightening mechanism is not operable to allow the spool to freely rotate within the interior region of the housing.

8. The device of claim 6, wherein the tightening mechanism is operable so that the spool is freely rotatable within the interior region of the housing.

9. A device for adjusting a fit of a brace about a limb comprising:
a tension member; and
a tightening mechanism that is operably coupled with the tension member such that the tension member forms a loop that is positionable about the brace to circumferentially surround an outer surface of the brace, the tightening mechanism being operable to decrease a diameter of the loop such that, after the loop is positioned about over the brace, the tension member constricts about the outer surface of the brace to tighten the brace about the limb.

10. The device of claim 9, wherein the tension member is uncoupled from any guide that is attached to the brace that guides or directs the tension member about a path.

11. The device of claim 9, further comprising a guide member that is coupled with the tightening mechanism to redirect the tension member and thereby define a second loop that is also positionable about the brace to circumferentially surround the outer surface of the brace, the tension member being slidably disposed within a channel of the guide member such that operation of the tightening mechanism causes a diameter of the second loop to decrease simultaneously with the decrease in the diameter of the loop.

12. The device of claim 9, wherein one end of the tension member is adjustably coupled with the tightening mechanism in a manner that allows the diameter of the loop to be decreased without operation of the tightening mechanism.

13. The device of claim 9, wherein a bottom surface of the tightening mechanism includes an adhesive that is bondable to an outer surface of the brace.

14. The device of claim 9, wherein the tightening mechanism is configured so that a first operation of the tightening mechanism decreases the diameter of the loop and thereby incrementally tightens the brace about the limb and so that a second operation of the tightening mechanism increases the diameter of the loop and thereby incrementally loosens the brace about the limb.

15. The device of claim 14, wherein the tightening mechanism includes a knob that is rotatable in a first direction to decrease the diameter of the loop and that is rotatable in a second direction to increase the diameter of the loop.

16. A method of adjusting a fit of a brace about a limb comprising:
for device comprising:
a tension member; and
a tightening mechanism that is operably coupled with the tension member such that the tension member forms a loop;
positioning the loop about the brace so that the tension member circumferentially surround an outer surface of the brace; and
operating the tightening mechanism to decrease a diameter of the loop and thereby constrict the tension member about the outer surface of the brace to tighten the brace about the limb.

17. The method of claim 16, wherein one end of the tension member is adjustably coupled with the tightening mechanism, and wherein the method further comprises adjusting the one end of the tension member relative to the tightening mechanism so that the diameter of the loop is decreased without operation of the tightening mechanism.

18. The method of claim 16, further comprising:
performing a first operation of the tightening mechanism to decrease the diameter of the loop and thereby incrementally tighten the brace about the limb; and
performing a second operation of the tightening mechanism to increase the diameter of the loop and thereby incrementally loosen the brace about the limb.

19. The method of claim 18, wherein performing the first operation of the tightening mechanism includes rotating a knob in a first direction and performing the second operation of the tightening mechanism includes rotating the knob in a second direction.

20. The method of claim 16, wherein the device further comprises a guide member that is coupled with the tightening mechanism to redirect the tension member and thereby define a second loop that is also positionable about the brace to circumferentially surround the outer surface of the brace, the tension member being slidably disposed within a channel of the guide member such that operation of the tightening mechanism causes a diameter of the second loop to decrease simultaneously with the decrease in the diameter of the loop.
